(19) United States
(12) Patent Application Publication
(10) Pub. No.: US 2017/0342110 A1
(43) Pub. Date: Nov. 30, 2017

(54) GLYCOPEPTIDES AND USES THEREOF

(71) Applicant: JAWAHARLAL NEHRU CENTRE FOR ADVANCED SCIENTIFIC RESEARCH (JNCASR), Bangalore (IN)

(72) Inventors: Jayanta HALDAR, Bangalore (IN); Venkateswarlu YARLAGADDA, Bangalore (IN)

(21) Appl. No.: 15/539,777

(22) PCT Filed: Dec. 24, 2015
(86) PCT No.: PCT/IN2015/050216

§ 371 Date: Jun. 26, 2017

(30) Foreign Application Priority Data
Dec. 25, 2014 (IN) 6565/CHE/2014

(51) Int. Cl. C07K 9/00; A61K 38/00

(52) U.S. Cl. CPC C07K 9/008; A61K 38/00

(57) ABSTRACT

The present disclosure provides a compound of Formula I or its stereoisomers, prodrugs and pharmaceutically acceptable salts thereof. The present disclosure also relates to process of preparation of vancomycin-sugar conjugates of Formula I, its stereoisomers, prodrugs, pharmaceutically acceptable salts thereof, and to pharmaceutical compositions containing them. The compounds of the present disclosure are useful in the treatment, prevention or suppression of diseases mediated by microbes.

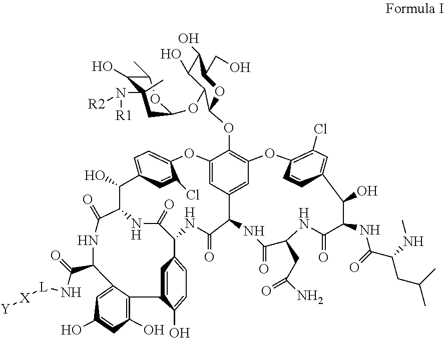

### FIELD OF INVENTION

The present disclosure relates to the field of medicinal chemistry and more particularly to the development of antibacterial compounds. The present disclosure particularly relates to vancomycin-sugar conjugates, its stereoisomers, prodrugs and pharmaceutically acceptable salts thereof. The present disclosure further relates to a process of preparing the vancomycin-sugar conjugates, its stereoisomers, prodrugs and pharmaceutically acceptable salts thereof. The present disclosure also relates to compositions and methods of treating conditions and diseases that are mediated by bacteria.

### BACKGROUND

Vancomycin, a glycopeptide antibiotic, has long been a drug of choice for life threatening gram-positive bacterial infections.^{1,2} Vancomycin inhibits bacterial cell wall synthesis by binding to the peptidoglycan peptide terminus D-Ala-D-Ala found in bacterial cell wall precursors, sequestering the substrate from transpeptidase and inhibiting cell wall cross-linking.^{2,3}

The alarming growth of antibiotic resistant superbugs such as vancomycin-resistant Enterococci and Staphylococci has become a major global health hazard. Vancomycin-resistant bacteria (VRB) developed resistance to vancomycin mainly by alteration of cell wall precursor; which leads to reduction in the binding constant of vancomycin to its target and subsequently results in loss of antibacterial activity.¹⁻³

Significant efforts have been directed toward the discovery of next-generation glycopeptide antibiotics that address the emerging drug-resistance of bacteria, especially vancomycin resistant strains.⁴⁻⁷ Thus, there is continuous need to identify and/or develop new compounds and/or derivatives that has enhanced activity against drug-resistant bacterial strains.

### SUMMARY

The present disclosure provides a compound of Formula I

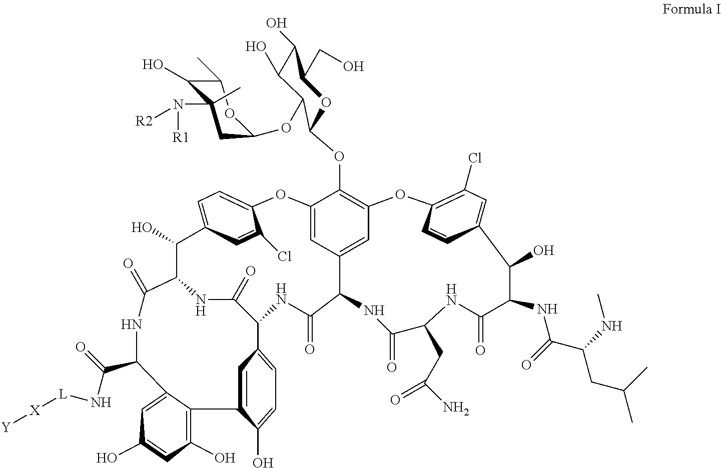

or its stereoisomers, prodrugs and pharmaceutically acceptable salts thereof:
wherein R¹ is selected from hydrogen, substituted or unsubstituted C₁-C₁₈ aliphatic radical or substituted or unsubstituted C₅-C₁₈ aromatic radical;
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;

### X is NH and O; and

Y is selected from the group consisting of cyclic monosaccharide, cyclic disaccharide, acyclic monosaccharide, acyclic disaccharide, and combinations thereof.

The present disclosure relates to a process for preparation of compound of Formula I or its stereoisomers, prodrugs and pharmaceutically acceptable salts thereof.

The present disclosure further relates to a compound of Formula I or its stereoisomers, prodrugs and pharmaceutically acceptable salts thereof, for use as a medicament.

The present disclosure also relates to a compound of Formula I or its stereoisomers, prodrugs and pharmaceutically acceptable salts thereof, for use in treatment of a bacterial infection.

The present disclosure relates to a pharmaceutical composition comprising a compound of Formula I or its stereoisomers, prodrugs and pharmaceutically acceptable salts thereof, together with a pharmaceutically acceptable carrier, optionally in combination with one or more other pharmaceutical compositions.

The present disclosure relates to a process for preparation of a pharmaceutical composition comprising a compound of Formula I or its stereoisomers, prodrugs and pharmaceutically acceptable salts thereof, together with a pharmaceutically acceptable carrier, optionally in combination with one or more other pharmaceutical compositions.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

The detailed description is described with reference to the accompanying FIGURES. In the FIGURES, the left-most digit(s) of a reference number identifies the FIGURE in which the reference number first appears. The same numbers are used throughout the drawings to reference like features and components.

FIG. 1 illustrates in-vivo antibacterial activity of a glycopeptide against MR-VISA.

### DETAILED DESCRIPTION

In the structural Formulae given herein and throughout the present disclosure, the following terms have been indicated meaning, unless specifically stated otherwise.

### Definitions

The term “alkyl” or “unsubstituted C₁-C₁₈ aliphatic radical” refers to a monoradical branched or unbranched saturated hydrocarbon chain having from 1 to 18 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” or “substituted C₁-C₁₈ aliphatic radical” refers to an alkyl group as defined above, having 1, 2, 3, or 4 substituents, preferably 1, 2 or 3 substituents, selected from the group consisting of alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy.

The term “alkenyl” refers to a monoradical of a branched or unbranched unsaturated hydrocarbon group preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 double bond (vinyl), preferably 1 double bond. Preferred alkenyl groups include ethenyl or vinyl (—CH═CH₂), 1-propylene or allyl (—CH₂CH═CH₂), isopropylene (—C(CH₃)═CH₂), bicyclo [2.2.1] heptene, and the like.

The term “substituted alkenyl” refers to an alkenyl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

The term “alkynyl” refers to a monoradical of an unsaturated hydrocarbon, preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 sites of acetylene (triple bond) unsaturation, preferably 1 triple bond. Preferred alkynyl groups include ethynyl, (—C≡CH), propargyl (or prop-1-yn-3-yl,—CH₂C≡CH), homopropargyl (or but-1-yn-4-yl, —CH₂CH₂C≡CH) and the like.

The term “substituted alkynyl” refers to an alkynyl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

“Halo” or “Halogen”, alone or in combination with any other term means halogens such as chloro (Cl), fluoro (F), bromo (Br) and iodo (I).

“Haloalkyl” refers to a straight chain or branched chain haloalkyl group with 1 to 6 carbon atoms. The alkyl group may be partly or totally halogenated. Representative examples of haloalkyl groups include but are not limited to fluoromethyl, chloromethyl, bromomethyl, difluoromethyl, dichloromethyl, dibromomethyl, trifluoromethyl, trichloromethyl, 2-fluoroethyl, 2-chloroethyl, 2-bromoethyl, 2,2,2-trifluoroethyl, 3-fluoropropyl, 3-chloropropyl, 3-bromopropyl and the like.

The term “aryl” or “unsubstituted C₅-C₁₈ aromatic radical” refers to an aromatic carbocyclic group of 5 to 18 carbon atoms having a single ring (e.g. phenyl) or multiple rings (e.g. biphenyl), or multiple condensed (fused) rings (e.g. naphthyl or anthranyl). Preferred aryls include phenyl, naphthyl and the like.

The term “substituted aryl” or “unsubstituted C₅-C₁₈ aromatic radical” refers to an aryl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy.

The term “arylalkyl” refers to an aryl group covalently linked to an alkylene group, where aryl and alkylene are defined herein.

The term “hydroxyalkyl” refers to the groups -alkylene-OH.

The term “carboxyalkyl” refers to the groups -alkylene-C(O)OH.

The term “cycloalkyl” refers to carbocyclic groups of from 3 to 20 carbon atoms having a single cyclic ring or multiple condensed rings which may be partially unsaturated. Such cycloalkyl groups include, by way of example, single ring structures such as cyclopropyl, cyclobutyl, cyclopentyl, cyclopentenyl, cyclohexyl, cyclohexenyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy.

“Cycloalkylalkyl” refers to an alkyl radical as defined above which is substituted by a cycloalkyl radical as defined above. Representative examples of cycloalkylalkyl include but are not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl and the like.

The term “heterocyclyl” refers to a saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl, pyrrolidinyl and the like.

The term “heterocyclylalkyl” refers to a heterocyclyl group covalently linked to an alkylene group, where heterocyclyl and alkylene are defined herein.

The term “heteroaryl” refers to an aromatic cyclic group having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring (if there is more than one ring). Such heteroaryl groups can have a single ring (e.g. pyridyl or furyl) or multiple condensed rings (e.g. indolizinyl, benzothiazolyl, or benzothienyl). Examples of heteroaryls include, but are not limited to, [1,2,4] oxadiazole, [1,3,4] oxadiazole, [1,3,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

“Glycopeptide’ refers to a heptapeptide antibiotics characterized by a multi-ring peptide core substituted with a saccharide groups.

“Saccharide’ refers to a simple sugar or a compound with sugars linked to each other. Saccharides are classified as mono-, di-, tri-, and polysaccharides according to the number of monosaccharide groups composing them.

The term “peptide” refers to a compound consisting of two or more amino acids linked in a chain, the carboxyl group of each acid being joined to the amino group

“Vancomycin” refers to the glycopeptide antibiotic having the structural Formula

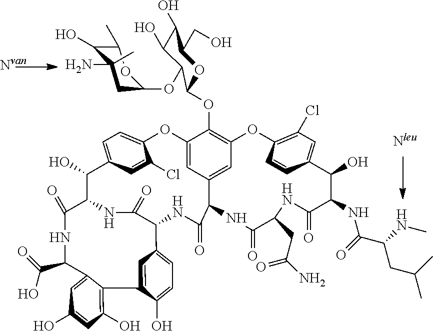

and is also represented in the disclosure by the Formula provided below:

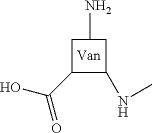

wherein —NH₂, —NHCH₃ represents N^{van}, and N^{leu} respectively.

Vancosamine moiety of vancomycin is shown as the N-site where a substituent can be covalently attached to the structure of Vancomycin.

The present disclosure provides a compound of Formula I

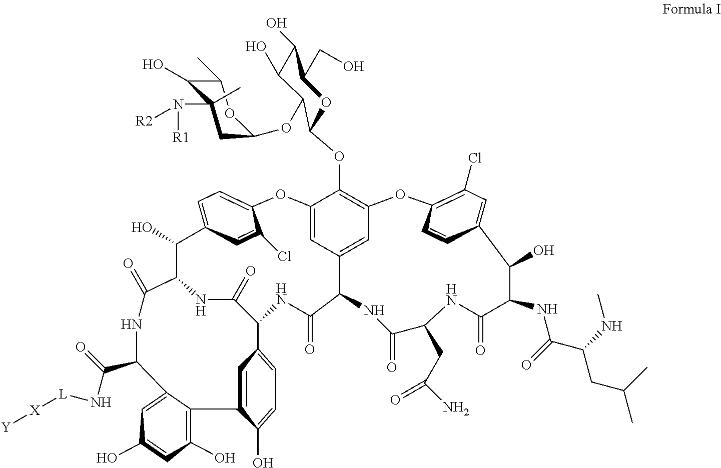

or its stereoisomers, prodrugs and pharmaceutically acceptable salts thereof:
wherein R¹ is selected from hydrogen, substituted or unsubstituted C₁-C₁₈ aliphatic radical or substituted or unsubstituted C₅-C₁₈ aromatic radical;
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;

### X is NH and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein R¹ is selected from hydrogen, substituted or unsubstituted C₁-C₁₈ aliphatic radical or substituted or unsubstituted C₅-C₁₈ aromatic radical;
R² is C₁-C₁₈ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;

### X is NH and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein R¹ is selected from hydrogen, substituted or unsubstituted C₁-C₁₈ aliphatic radical or substituted or unsubstituted C₅-C₁₈ aromatic radical;
R² is C₁-C₁₈ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;

### X is NH and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₉ aliphatic radical, substituted with trialkylamino, heteroaryl or heterocyclyl;
wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₃ aliphatic radical, substituted with trialkylamino, heteroaryl or heterocyclyl;
wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with trialkylamino, heteroaryl or heterocyclyl;
wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with trialkylamino, heteroaryl or heterocyclyl;
wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₉ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₃ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₉ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of Formula I or its stereoisomers, prodrugs and pharmaceutically acceptable salts thereof:

wherein
R¹ is hydrogen;
R² is C₁-C₃ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH, and O; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH; and

Y is lactobionic acid.

According to an embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₃ alkyl;

### X is NH, and O; and

Y is selected from the group consisting of cyclic monosaccharide, cyclic disaccharide, acyclic monosaccharide, acyclic disaccharide, and combinations thereof.

According to another embodiment, the present disclosure relates to compounds of Formula I or IA or its stereoisomers, prodrugs and pharmaceutically acceptable salts thereof:

wherein Y is selected from the group consisting of

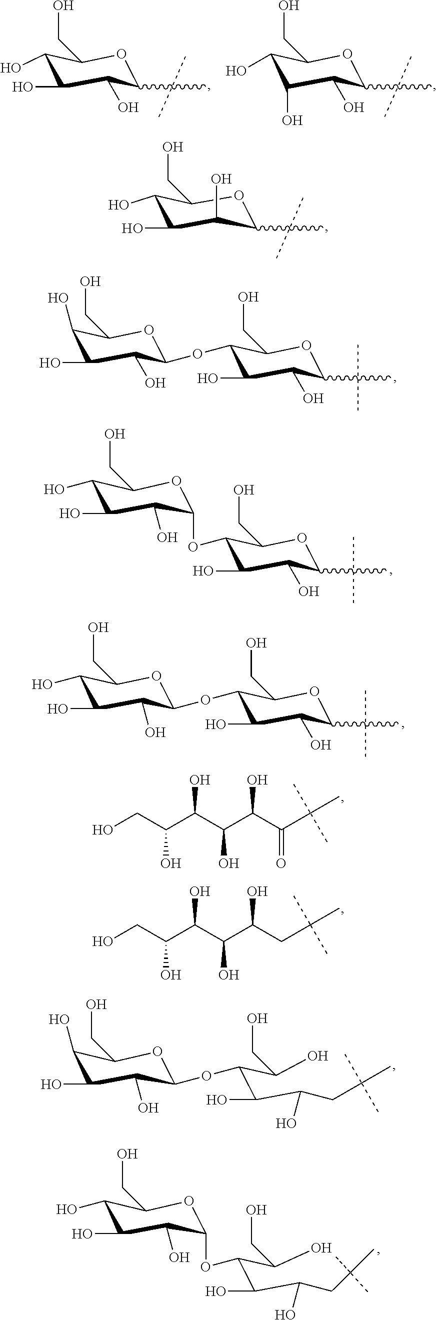

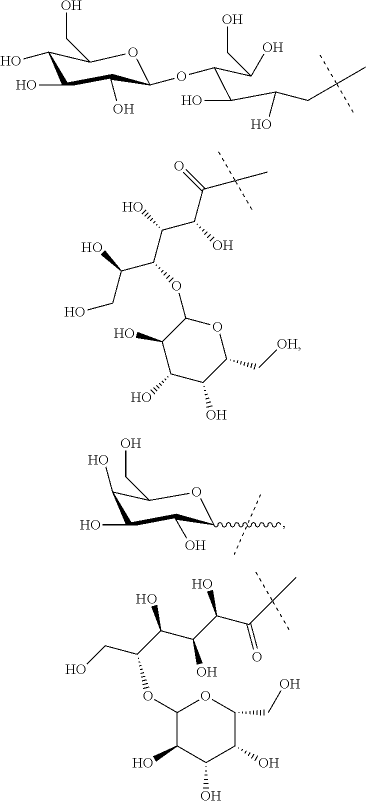

According to another embodiment, the present disclosure relates to compounds of Formula I or IA or its stereoisomers, prodrugs and pharmaceutically acceptable salts thereof:

wherein Y is selected from the group consisting of

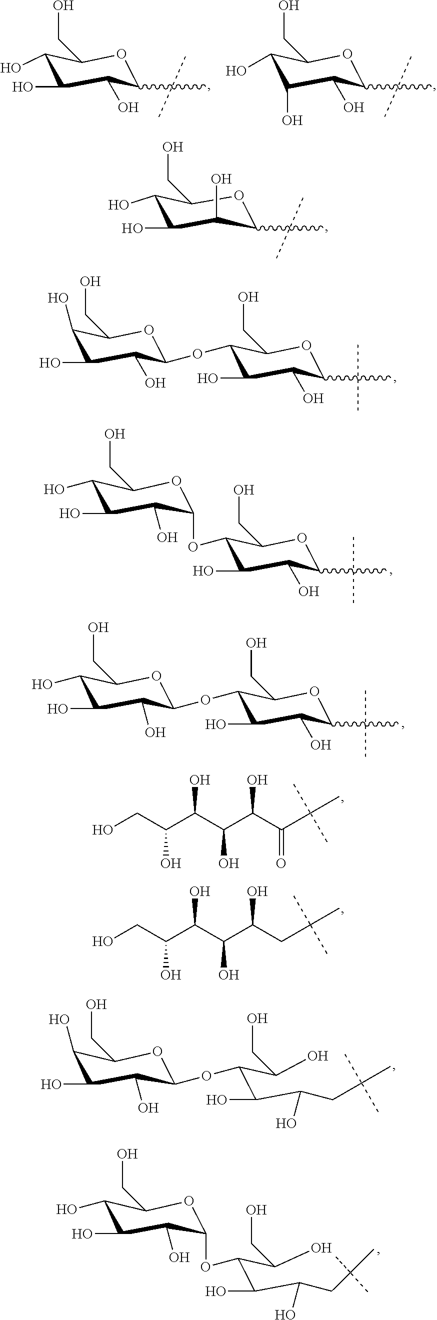

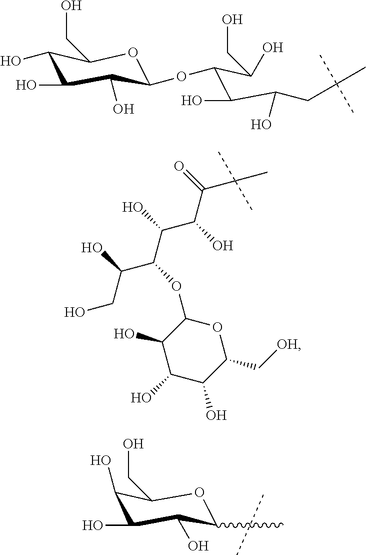

According to yet another embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of

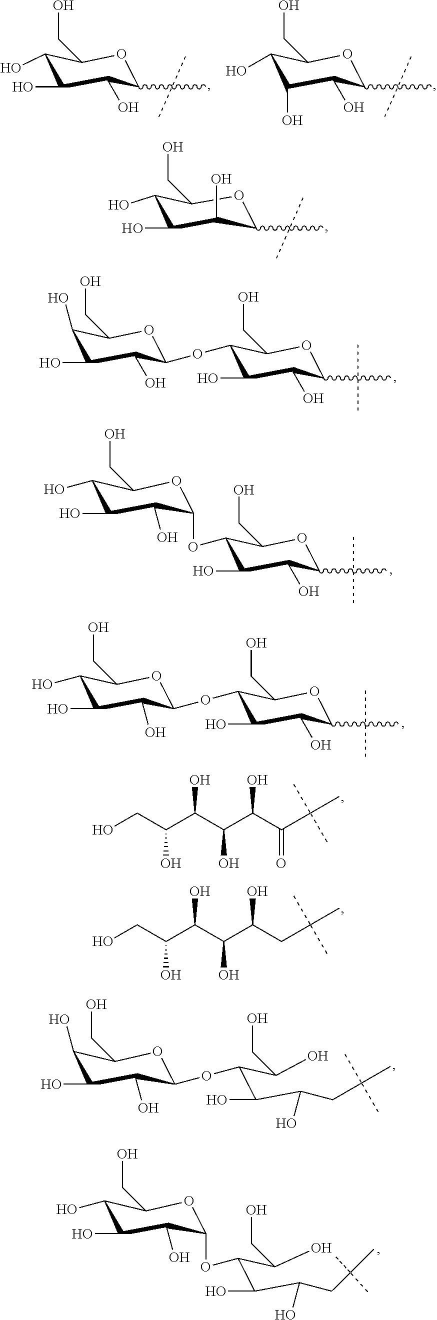

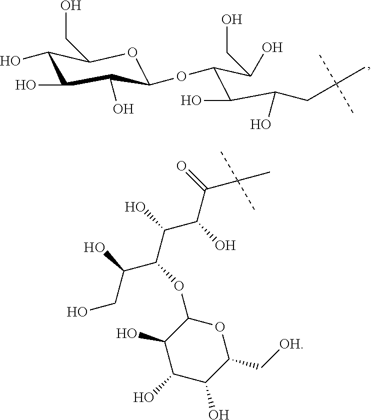

According to an embodiment, the present disclosure relates to compounds of Formula I or its stereoisomers, prodrugs and pharmaceutically acceptable salts thereof:

wherein
R¹ is hydrogen;
R² is C₁-C₉ aliphatic radical, substituted with heteroaryl; wherein heteroaryl is independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of

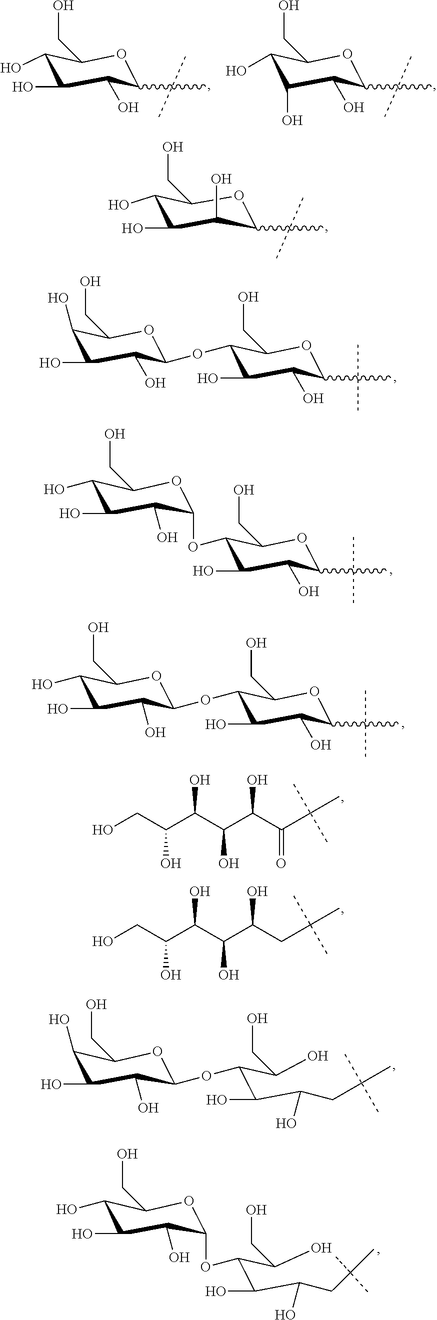

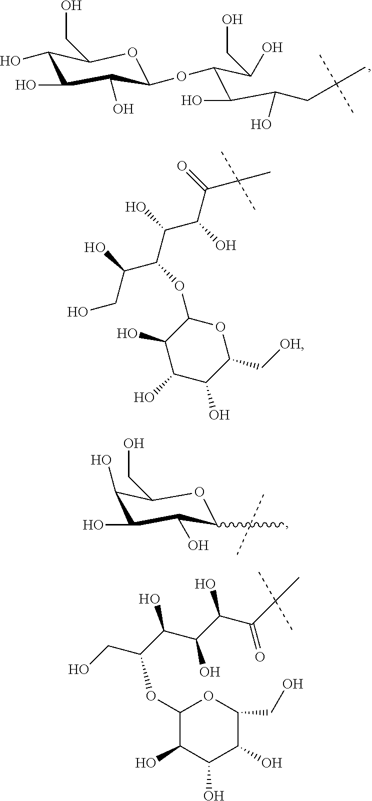

According to another embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of

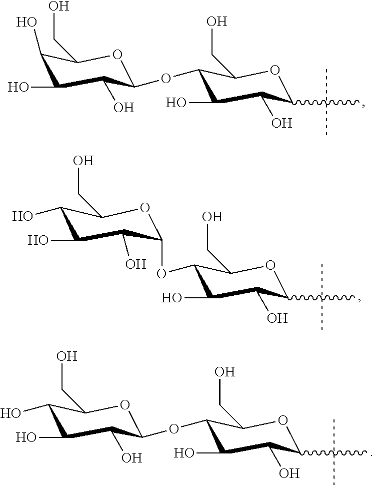

According to yet another embodiment, the present disclosure relates to compounds of Formula I or its stereoisomers, prodrugs and pharmaceutically acceptable salts thereof:

wherein
R¹ is hydrogen;
R² is C₁-C₃ aliphatic radical, substituted with heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O; and

Y is selected from the group consisting of

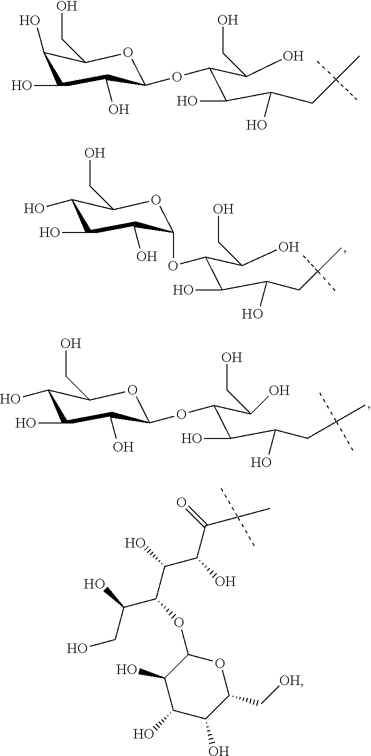

According to another embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with trialkylamino;
L is a C₂-C₆ alkyl;

### X is NH, or O;

Y is selected from the group consisting of

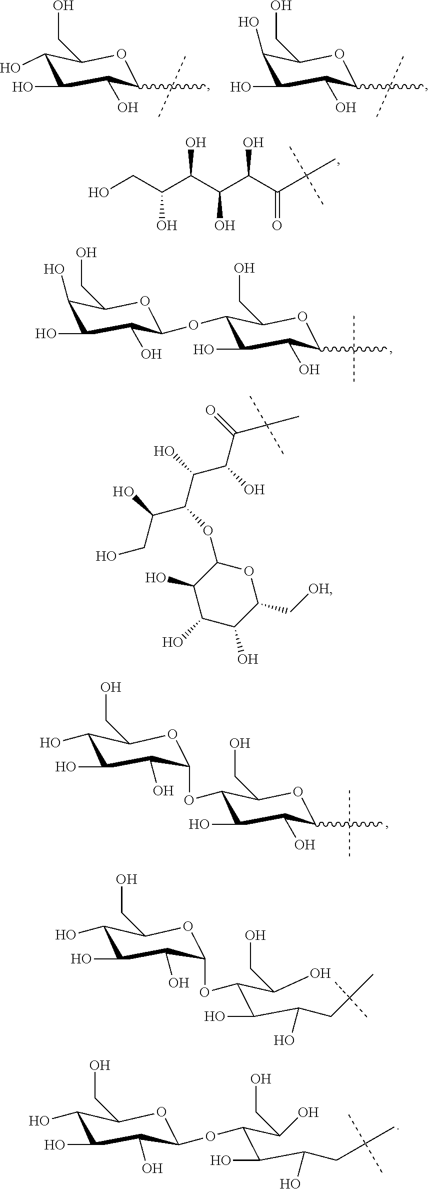

According to an embodiment, the present disclosure relates to compounds of Formula I or its stereoisomers, prodrugs and pharmaceutically acceptable salts thereof:

wherein
R¹ is hydrogen;
R² is C₁-C₁₈ aliphatic radical, substituted with heterocyclyl; wherein heterocyclyl is independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is O; and

Y is selected from the group consisting of

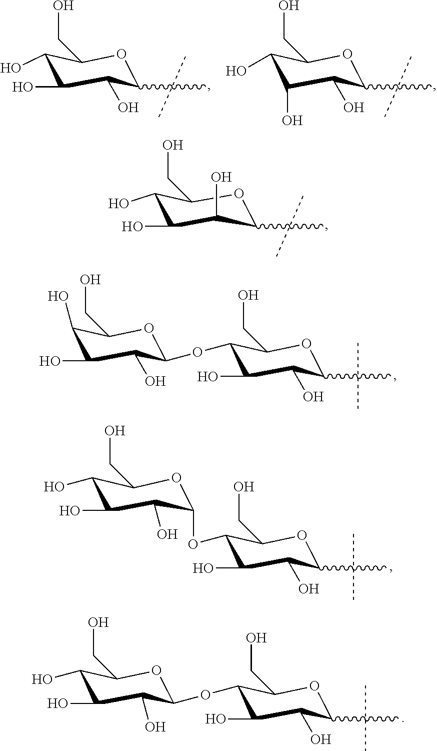

According to another embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH; and

Y is selected from the group consisting of

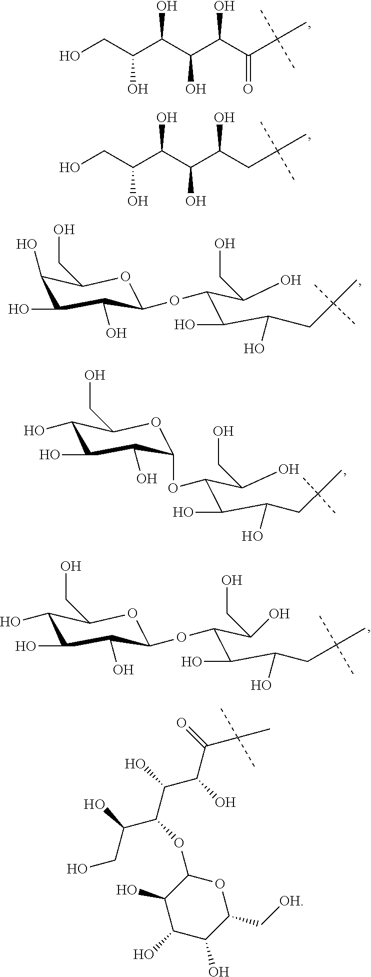

According to yet another embodiment, the present disclosure relates to compounds of Formula I or its stereoisomers, prodrugs and pharmaceutically acceptable salts thereof:

wherein
R¹ is hydrogen;
R² is C₁ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH; and

Y is selected from the group consisting of

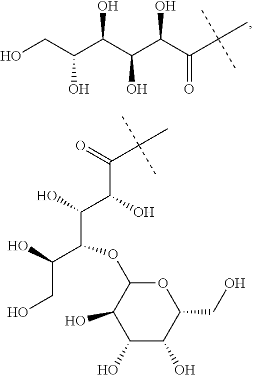

According to another embodiment, the present disclosure relates to compounds of Formula I or its stereoisomers, prodrugs and pharmaceutically acceptable salts thereof:

wherein R¹ is hydrogen,
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O;

Y is selected from the group consisting of

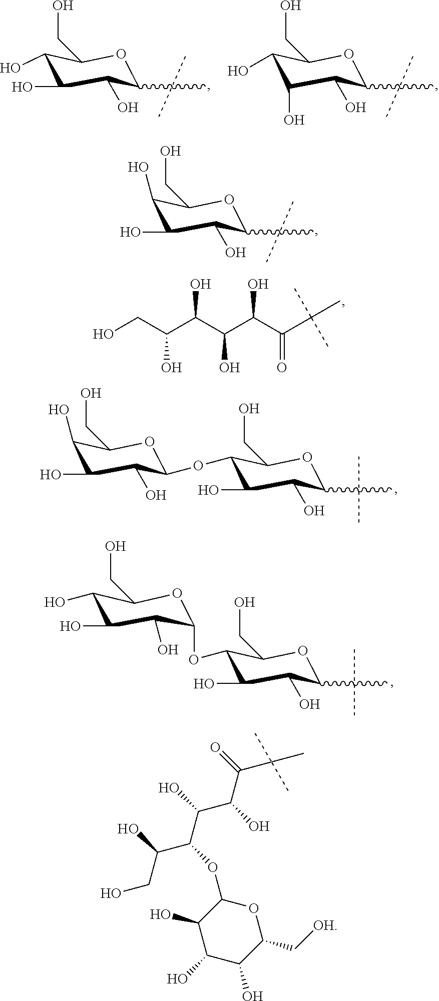

According to another embodiment, the present disclosure relates to compounds of Formula I or its stereoisomers, prodrugs and pharmaceutically acceptable salts thereof:

wherein R¹ is hydrogen,
R² is C₁-C₃ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is a C₂-C₆ alkyl;

### X is NH, and O;

Y is selected from the group consisting of

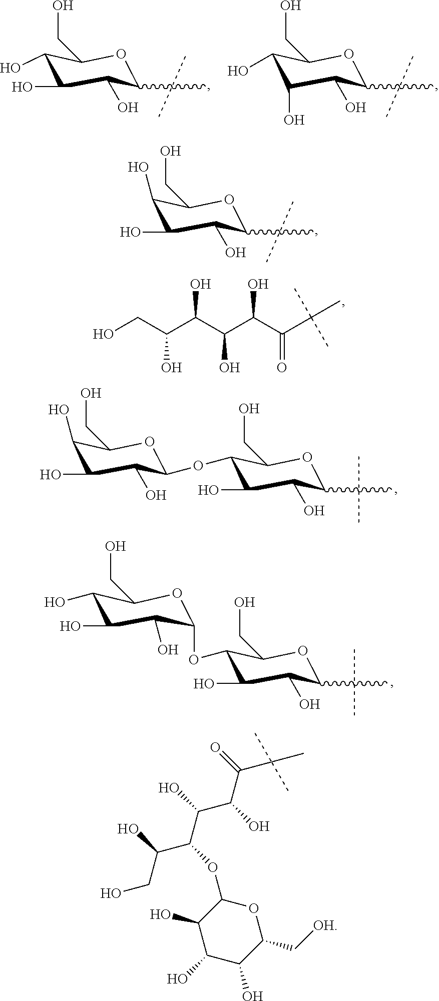

According to an embodiment, the present disclosure relates to compounds of Formula IA

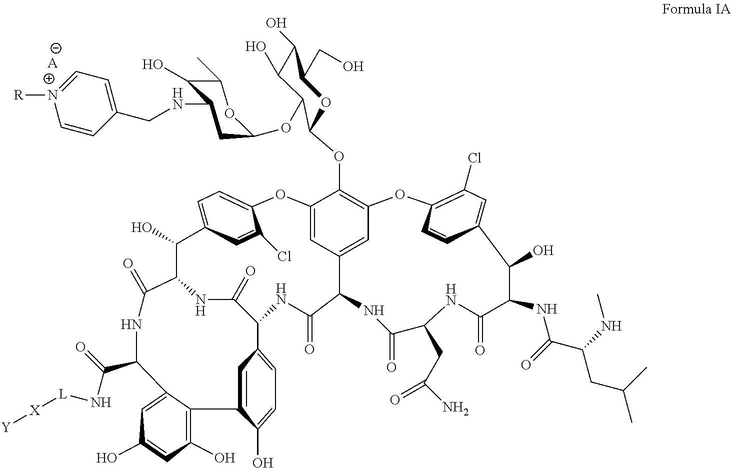

or its stereoisomers, prodrugs and pharmaceutically acceptable salts thereof:
wherein
R is selected from hydrogen, substituted or unsubstituted C₂-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;

### X is NH and O; and

Y is selected from the group consisting of cyclic monosaccharide, cyclic disaccharide, acyclic monosaccharide, acyclic disaccharide, and combinations thereof;

A^{⊖} is negatively charged counter anion.

According to an embodiment, the present disclosure relates to compounds of Formula IA or its stereoisomers, prodrugs and pharmaceutically acceptable salts thereof:

wherein R is selected from the group consisting of hydrogen, a C₂-C₁₈ alkyl, a C₅-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl,

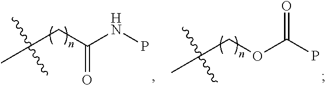

wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy; P is a C₂-C₁₈ aliphatic radical and n is an integer ranging from 1 to 4, L is a C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH and O;

Y is selected from the group consisting of cyclic monosaccharide, cyclic disaccharide, acyclic monosaccharide, acyclic disaccharide, and combinations thereof; and
A^{⊖} is negatively charged counter anion.

According to an embodiment, the present disclosure relates to compounds of Formula IA or its stereoisomers, prodrugs and pharmaceutically acceptable salts thereof:

wherein
A^{⊖} is negatively charged counter anion selected from the group consisting of Cl⁻, Br⁻, I⁻, OH⁻, HCO₃⁻, CO₃²⁻, R₃COO⁻, R₃SO₄⁻, and R₃SO₃⁻;
R₃ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl.

According to an embodiment, the present disclosure relates to compounds of Formula IA or its stereoisomers, prodrugs and pharmaceutically acceptable salts thereof:

R is selected from the group consisting of hydrogen, substituted or unsubstituted C₂-C₁₈ aliphatic radical and substituted or unsubstituted C₅-C₁₈ aromatic radical.
L is substituted or unsubstituted C₂-C₁₈ aliphatic radical;

### X is NH;

Y is selected from the group consisting of

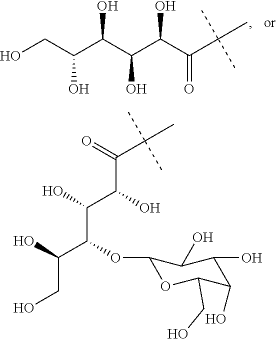

According to an embodiment, the present disclosure relates to compounds of Formula IA or its stereoisomers, prodrugs and pharmaceutically acceptable salts thereof:

wherein

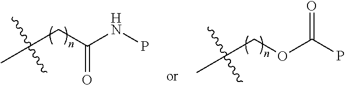

R is selected from or
L is substituted or unsubstituted C₂-C₁₈ aliphatic radical;

### X is NH;

Y is selected from the group consisting of

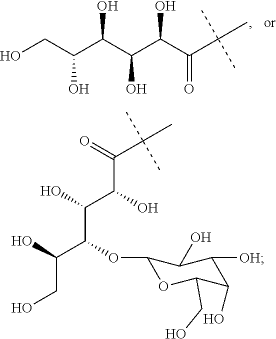

P is a C₂-C₁₈ aliphatic radical and n is an integer ranging from 1 to 4.

According to an embodiment, the present disclosure relates to compounds of Formula IA or its stereoisomers, prodrugs and pharmaceutically acceptable salts thereof:

wherein
R is substituted or unsubstituted C₅-C₁₈ aliphatic radical;
L is substituted or unsubstituted C₃ aliphatic radical;

### X is NH; and

Y is selected from the group consisting of

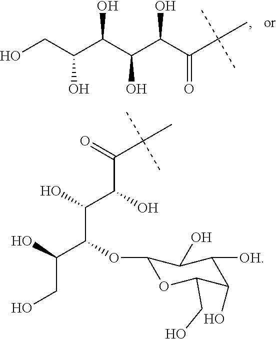

According to an embodiment, the present disclosure relates to compounds of Formula IA or its stereoisomers, prodrugs and pharmaceutically acceptable salts thereof:

wherein
R is selected from the group consisting of hydrogen, a C₂-C₁₈ alkyl, a C₅-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl,

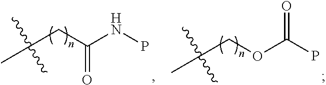

wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy; L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH;

Y is selected from the group consisting of

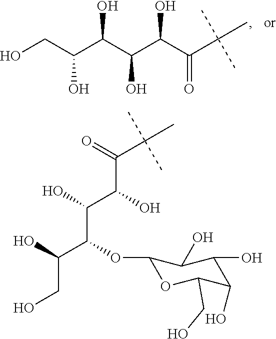

According to an embodiment, the present disclosure relates to compounds of Formula IA or its stereoisomers, prodrugs and pharmaceutically acceptable salts thereof:

wherein
R is selected from or

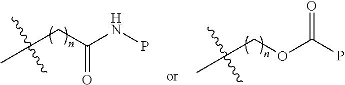

L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH;

Y is selected from the group consisting of

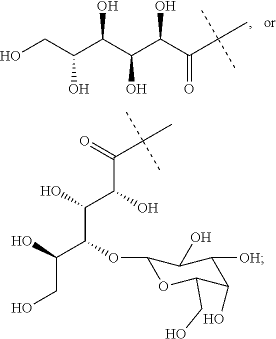

and
P is a C₂-C₁₈ aliphatic radical and n is an integer ranging from 1 to 4.

According to an embodiment, the present disclosure relates to compounds of Formula IA or its stereoisomers, prodrugs and pharmaceutically acceptable salts thereof:

wherein
R is C₆-C₁₈ alkyl;
L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH; and

Y is selected from the group consisting of

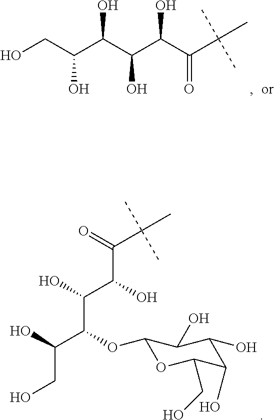

One embodiment of the present disclosure are compounds of Formula I or its stereoisomers, prodrugs and pharmaceutically acceptable salts thereof, selected from the group consisting of,

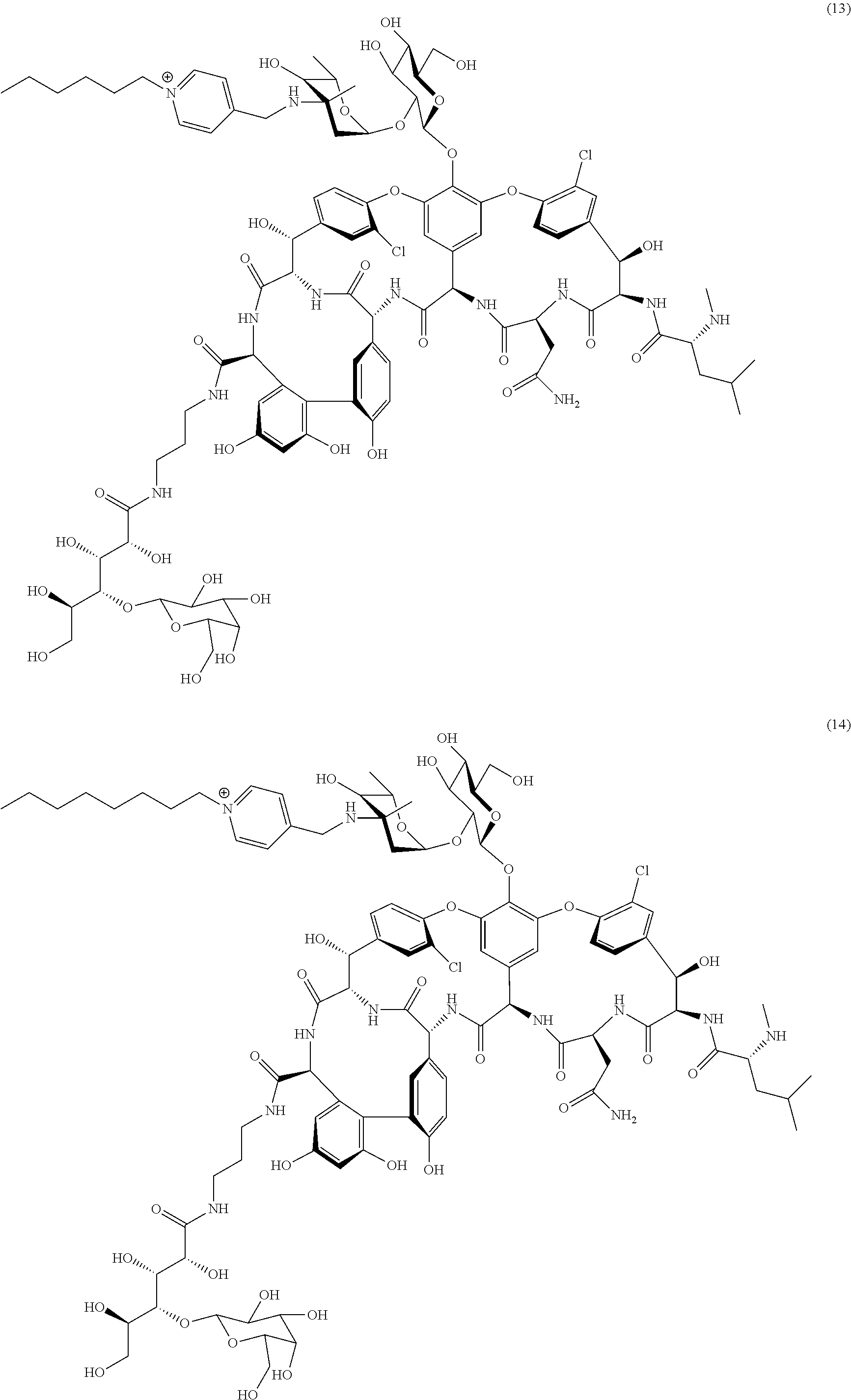

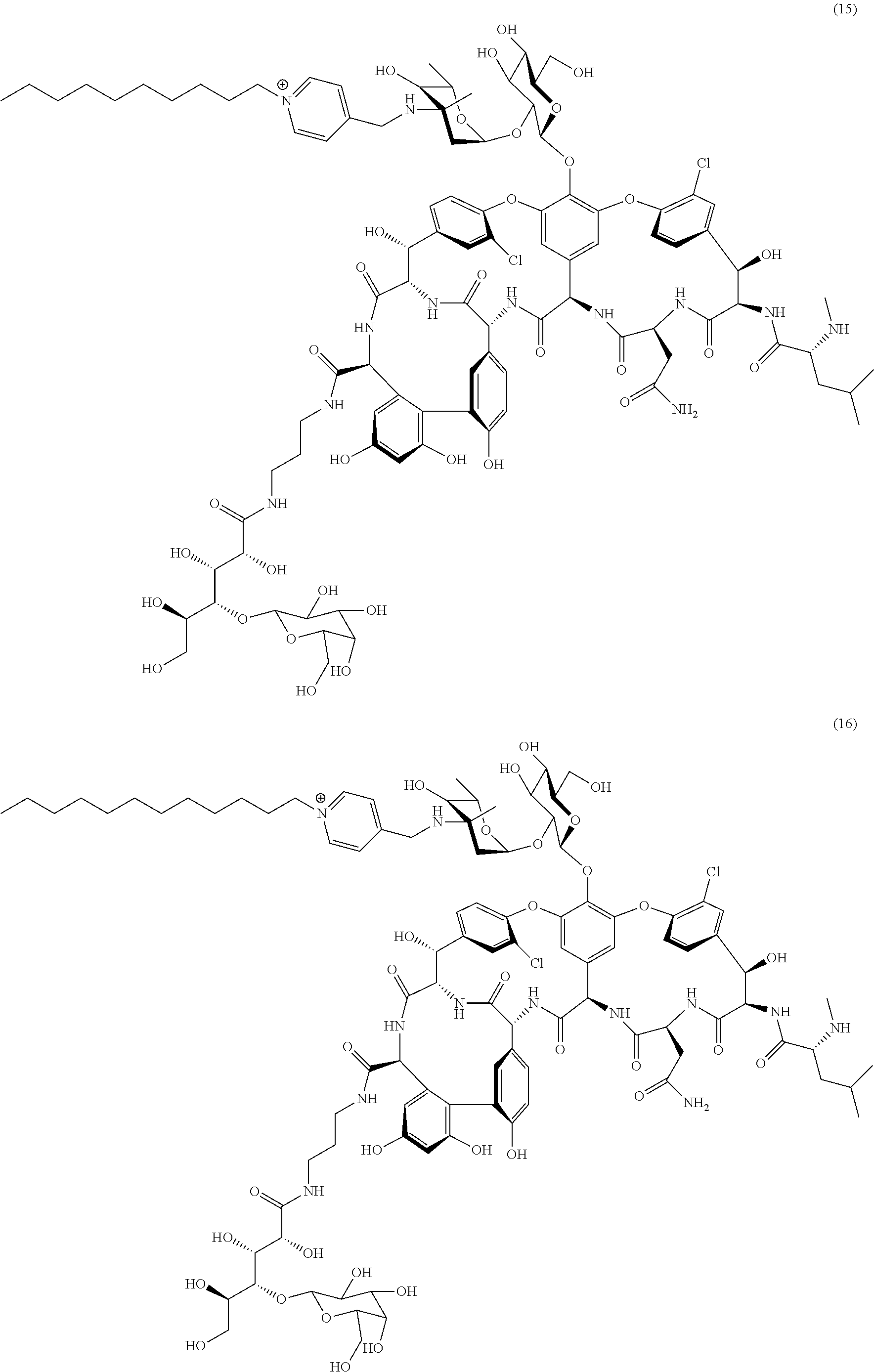

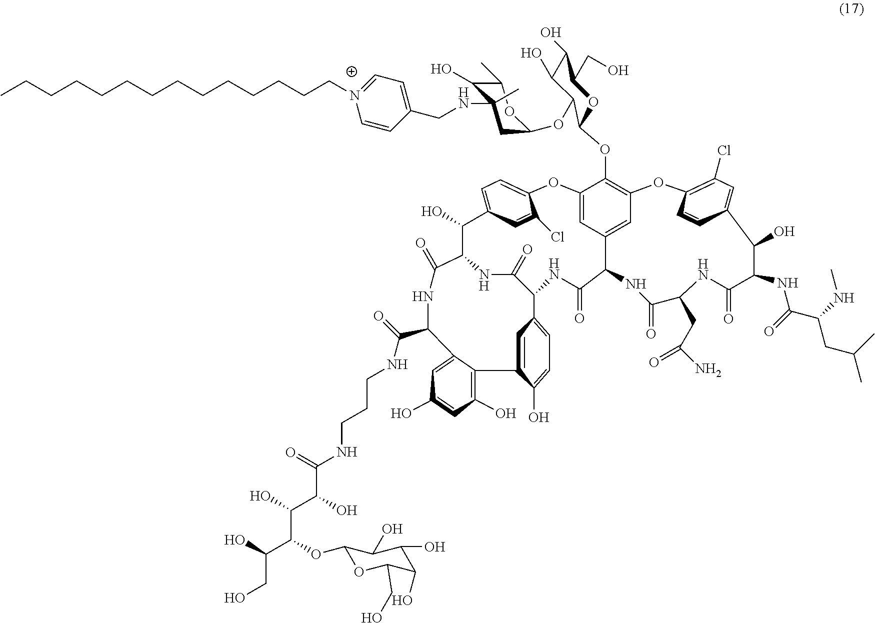

An embodiment of the present disclosure also relates to a compound of Formula (I) or IA or its stereoisomers, prodrugs and pharmaceutically acceptable salts thereof, for use as a medicament.

Another embodiment of the present disclosure also relates to a compound of Formula (I) or IA or its stereoisomers, prodrugs and pharmaceutically acceptable salts thereof, for use in treatment of a bacterial infection.

Yet another embodiment of the present disclosure also relates to a compound of Formula (I) or IA or its stereoisomers, prodrugs and pharmaceutically acceptable salts thereof, for use in the treatment of diseases caused by gram positive bacteria.

Another embodiment of the present disclosure relates to a pharmaceutical composition comprising a compound of Formula (I) or IA or pharmaceutically acceptable salts thereof, together with a pharmaceutically acceptable carrier and a method of preparing the same.

Yet another embodiment of the present disclosure relates to a pharmaceutical composition comprising a therapeutically effective amount of a compound of the present disclosure, alone or in combination with one or more pharmaceutically acceptable carriers.

An embodiment of the present disclosure relates to a method of killing a bacterial cell, the method comprising contacting the cell with a compound of Formula (I) or IA or its stereoisomers, prodrugs and pharmaceutically acceptable salts thereof, in an amount sufficient to kill the bacterial cell.

In an embodiment of the present disclosure the bacterial cell is selected from the group consisting of enterococci, staphylococci and streptococci.

The present disclosure describes vancomycin-sugar conjugates using facile synthetic methodology. These derivatives showed strong, broad-spectrum antibacterial activity and about >7000 fold more active than parent drug, vancomycin against vancomycin-resistant E. faecium (VRE). The compounds are highly bactericidal and show excellent in-vivo antibacterial activity against vancomycin resistant bacterial infection with improved pharmacological properties. In fact, the disclosed glycopeptide is more effective than the comparator drugs such as vancomycin and linezolid. These compounds are the first examples of a new generation of glycopeptide antibiotics that can be developed to tackle vancomycin-resistant enterococcal infections.

An embodiment of the present disclosure also relates to a compound of Formula (I) or IA or its stereoisomers, prodrugs and pharmaceutically acceptable salts thereof, for use in treatment of a bacterial infection, wherein the bacterium comprises a vancomycin-resistant bacterium or a methicillin-resistant bacterium.

An embodiment of the present disclosure also relates to a compound of Formula (I) or IA or its stereoisomers, prodrugs and pharmaceutically acceptable salts thereof, for use in treatment of a bacterial infection, wherein the bacterium comprises a vancomycin-resistant Staphylococcus aureus, a vancomycin-resistant Enterococcus faecium or a methicillin-resistant Staphylococcus aureus.

Another embodiment of the disclosure includes a method of treatment of bacterial infection in a subject by administering to the subject an effective amount of the compound of Formula I or IA or its stereoisomers, prodrugs and pharmaceutically acceptable salts thereof.

The bacterial infection disclosed in the present disclosure is caused by a gram-positive bacterium.

The bacterial infection comprises an infection caused by a drug-resistant bacterium. The drug-resistant bacterium of the present disclosure is a vancomycin-resistant bacterium or a methicillin-resistant bacterium. The bacterium comprises a vancomycin-resistant Staphylococcus aureus, a vancomycin-resistant Enterococcus faecium or a methicillin-resistant Staphylococcus aureus.

A further embodiment of the present disclosure also relates to an article comprising: a composition comprising the compound of Formula I or IA or its stereoisomers, prodrugs and pharmaceutically acceptable salts thereof.

In an embodiment, the article comprises a substrate, wherein the substrate is coated with or impregnated with the composition comprising the compound of Formula I or IA or its stereoisomers, prodrugs and pharmaceutically acceptable salts thereof.

The present disclosure further relates to a process of preparation of compounds of Formula (I) or IA or stereoisomers, prodrugs and pharmaceutically acceptable salts thereof.

The present subject matter further discloses a process for the preparation of vancomycin sugar conjugates of Formula I or IA. In an embodiment, the sugar conjugates of vancomycin of the present subject matter were synthesized by coupling carboxylic group of vancomycin with cyclic, acyclic sugar moieties, or combinations thereof through amide coupling using at least one organic solvent and coupling agent. Further, the reaction is carried out between 0° C.-room temperature. In one embodiment, the coupling agent is o-benzotriazole-N,N,N′,N′-tetramethyl-uronium-hexafluorophosphate (HBTU). Other coupling agents such as 2-(1H-7-azabenzotriazol-1-yl)-1,1,3,3-tetramethyl uronium hexafluorophosphate Methanaminium (HATU), N,N′-diisopropylcarbodiimide (DIC), 1-ethyl-3-(3-dimethylaminopropyl carbodiimide (EDCI) and O-(benzotriazol-1-yl)-N,N,N′,N′-tetramethyluronium tetrafluoroborate (TBTU) can be used instead of HBTU. The reaction mixture should be cooled to 0° C., and 1.5 equivalents of amide coupling reagent (HBTU) in DMF should be added followed by 5.0 equivalents of diisopropylethylamine (DIPEA) and then appropriate amine should be added for amide coupling. The reaction mixture was then allowed to warm to room temperature (25° C.) and stirred for 8-12 h. In another embodiment, the organic solvent includes at least one selected from the group of dimethylformamide (DMF), dimethyl sulfoxide, and others as understood by a person skilled in the art.

In an embodiment, the synthesized compounds are further characterized by IR, ¹H-NMR, ¹³C-NMR and HR-MS.

### Abbreviations

The following abbreviations are employed in the examples and elsewhere herein:

### DCM: Dichloromethane,

NaN₃: Sodium azide,

### CH₃OH: Methanol,

NaOMe: Sodium methoxide,
PPh₃: Triphenyl phosphine,

### DMF: N,N-Dimethylformamide,

DMSO: Dimethyl sulfoxide,
HBTU: Benzotriazole-N,N,N′,N′-tetramethyl-uronium-hexafluorophosphate,

### DIPEA: Diisopropylethylamine,

HCl: Hydrochloric acid,

### IPA: Isopropanol,

NaBH₄: Sodium borohydride,
NaCNBH₃: Sodium cyanoborohydride
RT: Room temperature,

### μM: Micromolar.

### EXAMPLES

The following examples provide details concerning the synthesis, activities, and applications of the compounds of the present disclosure. It should be understood the following is representative only, and that the invention is not limited by the details set forth in these examples.

All reagents were purchased from Sigma-Aldrich and SD Fine and used without further purification. Analytical thin layer chromatography (TLC) was performed on E. Merck TLC plates pre-coated with silica gel 60 F₂₅₄ (250 μm thickness). Visualization was accomplished using UV light, Potassium permangante and Iodine. Column chromatography was performed on silica gel (60-120 A° pore size). HPLC analysis was performed on a Shimadzu-LC 8A Liquid Chromatograph instrument (C₁₈ column, 10 mm diameter, 250 mm length) with UV detector monitoring at 270 nm. Nuclear magnetic resonance spectra were recorded on Bruker (AV-400) 400 MHz spectrometer in deuterated solvents. Mass spectra were obtained using 6538-UHD Accurate mass Q-TOF LC-MS instrument. Bacterial strains, S. aureus MTCC 737 was purchased from MTCC (Chandigarh, India) and MRSA ATCC 33591, E. faecalis ATCC 51575 and E. faecium ATCC 51559 were obtained from ATCC (Rockville, Md.). Tryptic-soy agar media was used for Staphylococci and Enterococci. Eppendorf 5810R centrifuge was used. TECAN (Infinite series, M200 pro) Plate Reader was used to measure absorbance. CD-1 or BALB/c mice were used for in-vivo studies.

### Example 1

Cationic-lipophilic-vancomycin-sugar conjugates (13-17) (Scheme 1) of the instant disclosure were synthesized by coupling carboxylic group of Cationic-lipophilic-vancomycin analogues (6-10) (Scheme 1) with cyclic and acyclic sugar moiety (12) through amide coupling using O-benzotriazole-N,N,N′,N′-tetramethyl-uronium-hexafluorophosphate (HBTU) (Scheme 2-5). Initially lipophilic-cationic moieties (1-5) were coupled to vancomycin through Schiffs base formation to give cationic-lipophilic-vancomycin analogues (6-10). The steps employed in the method of synthesizing sugar moiety and cationic lipophilic moieties are further elaborated below in Examples 1.1-1.4.

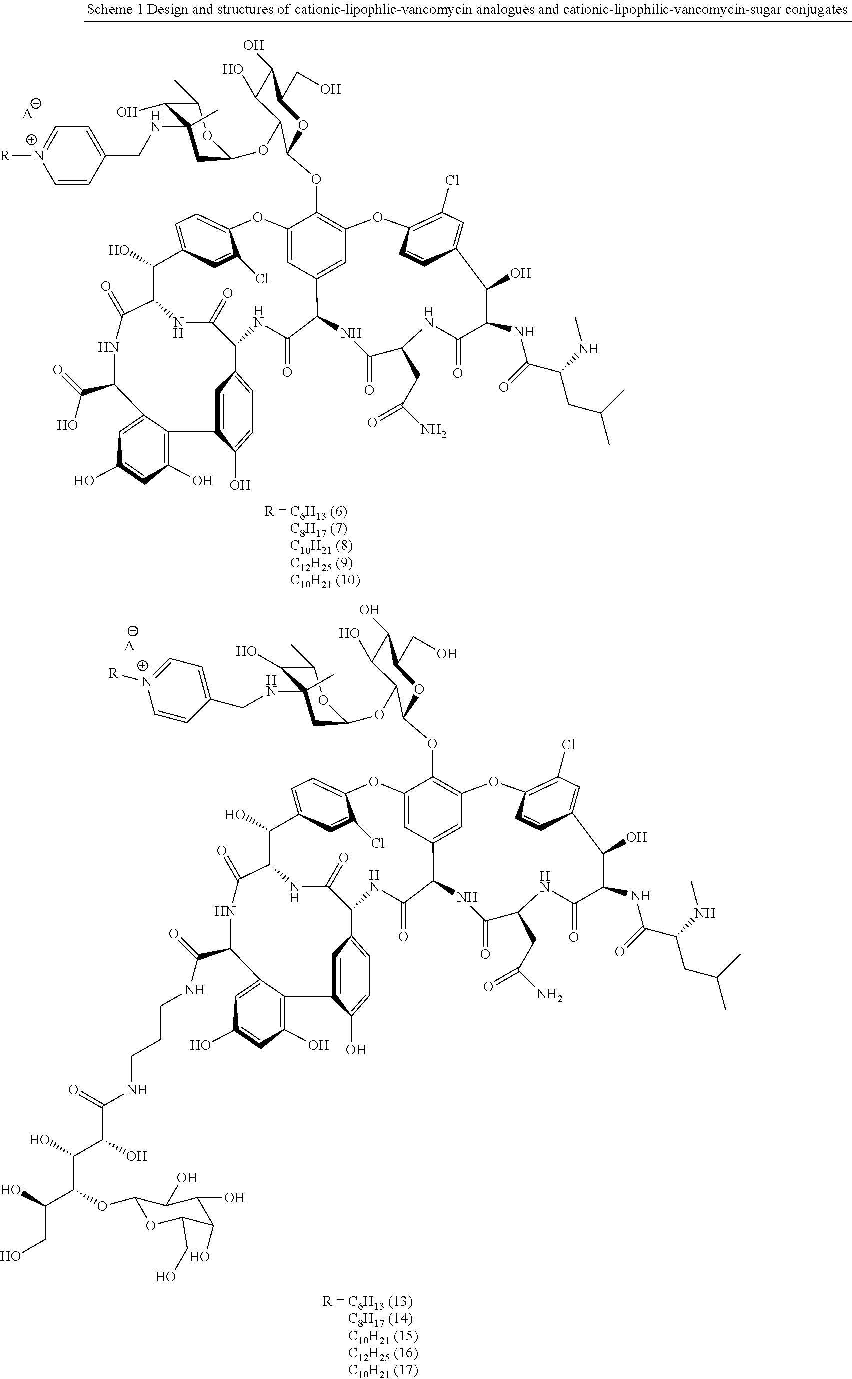

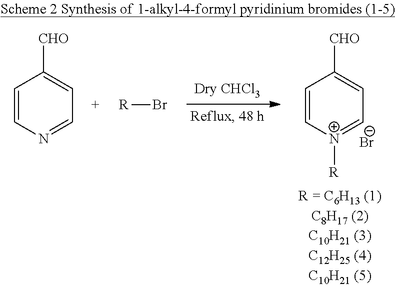

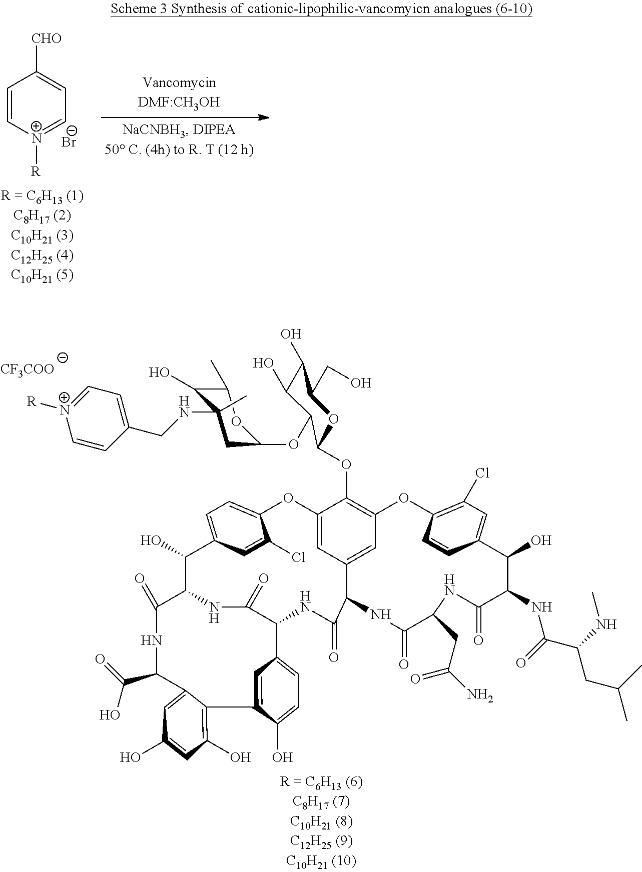

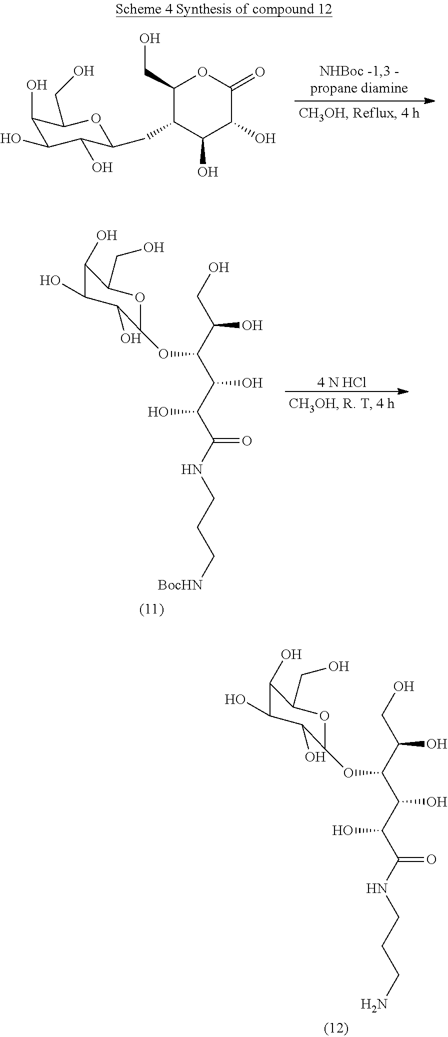

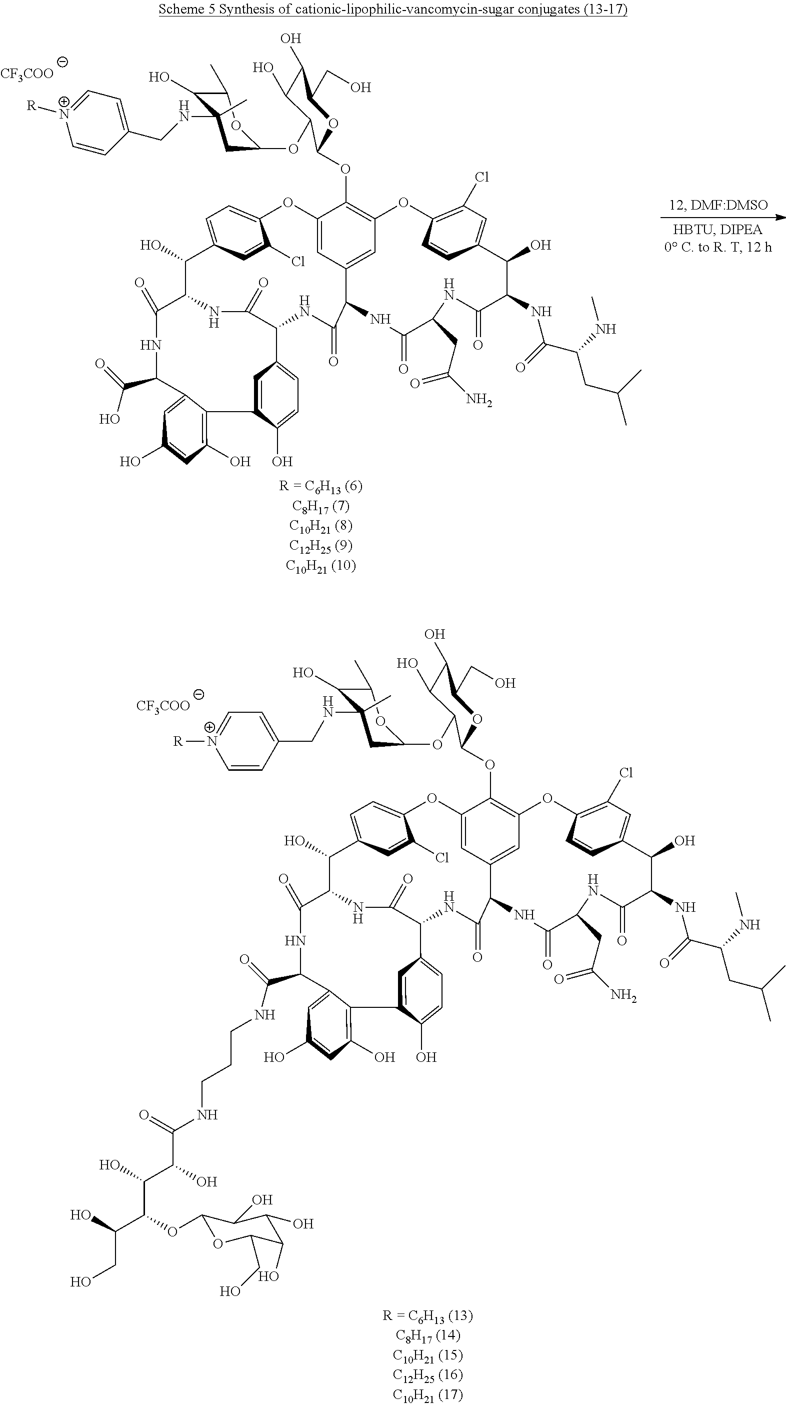

### Example 1.1: Synthesis of 1-alkyl-4-formyl pyridinium bromides (1-5)

About 1.0 g of Pyridine-4-aldehyde was taken in 15 mL dry chloroform and about 2 equivalents of alkyl bromide (R-Br, R varied from hexyl to tetradecyl) was added to it and then refluxed for 48 h. For this reaction, dry chloroform can be substituted with a non polar solvent, such as dichloromethane or a polar solvent, such as ethyl acetate. Then the reaction was allowed to come to room temperature and the solvent was evaporated. The crude product was washed with dry hexane and followed by dry ethyl acetate. The traces of the solvent were removed by using high vacuum pump to afford 1-alkyl-4-formyl pyridinium bromides (1-5) in 35 to 40% yield.

Compound 1: ¹H-NMR (400 MHz, CDCl₃) δ/ppm: 10.51 (s, 1H), 9.18-9.09 (d, 2H), 8.31-8.2 (d, 2H), 4.91-4.83 (t, 2H), 2.2-2.11 (m, 2H), 1.35-1.21 (m, 10H), 0.89-0.86 (t, 3H).

Compound 2: ¹H-NMR (400 MHz, CDCl₃) δ/ppm: 10.42 (s, 1H), 9.08-9.06 (d, 2H), 8.32-8.17 (d, 2H), 4.80-4.75 (t, 2H), 2.17-2.01 (m, 2H), 1.33-1.23 (m, 10H), 0.87-0.84 (t, 3H).

Compound 3: ¹H-NMR (400 MHz, CDCl₃) δ/ppm: 10.11 (s, 1H), 9.07-9.00 (d, 2H), 8.30-8.16 (d, 2H), 4.73-4.72 (t, 2H), 2.08-2.00 (m, 2H), 1.33-1.23 (m, 14H), 0.88-0.85 (t, 3H).

Compound 4: ¹H-NMR (400 MHz, CDCl₃) δ/ppm: 10.09 (s, 1H), 9.08-9.06 (d, 2H), 8.30-8.17 (d, 2H), 4.77-4.75 (t, 2H), 2.08-2.02 (m, 2H), 1.35-1.24 (m, 18H), 0.88-0.85 (t, 3H).

Compound 5: ¹H-NMR (400 MHz, CDCl₃) δ/ppm: 10.19 (s, 1H), 9.05-9.02 (d, 2H), 8.22-8.15 (d, 2H), 4.67-4.61 (t, 2H), 2.11-2.07 (m, 2H), 1.37-1.25 (m, 22H), 0.89-0.86 (t, 3H).

### Example 1.2: Synthesis of cationic-lipophilic-vancomycin analogues (6-10)

Vancomycin hydrochloride (150 mg) was dissolved in dry dimethyl formamide (1 mL) and dry methanol (1 mL). To this 1.5 equivalents of 1-alkyl-4-formyl pyridinium bromides (1-5) and 1.2 equivalents of diisopropylethylamine (DIPEA) were added. The reaction mixture was stirred at 50° C. for 2 h and then allowed to cool to room temperature prior to addition of sodium cyanoborohydride (2.0 equivalents). Then, the reaction mixture was stirred at 50° C. for additional 2 h and allowed to cool to ambient temperature for overnight. The product was purified by preparative reversed-phase HPLC using 0.1% trifluoroacetic acid in H₂O/acetonitrile mixture and then lyophilized to afford trifluoroacetate salt compounds 6 to 10 in 60-75% yield.

Compound 6: Yield; 63%. HR-MS: m/z 813.2645 (observed), 813.2632 (calculated for M+H)²⁺.

Compound 7: Yield; 71%. HR-MS: m/z 827.3086 (observed), 827.2895 (calculated for M+H)²⁺.

Compound 8: Yield; 75%. HR-MS: m/z 841.3186 (observed), 841.3158 (calculated for M+H)²⁺.

Compound 9: Yield; 69%. HR-MS: m/z 855.3396 (observed), 855.3421 (calculated for M+H)²⁺.

Compound 10: Yield; 65%. HR-MS: m/z 869.8133 (observed), 869.3684 (calculated for M+H)²⁺.

### Example 1.3

### Example 1.3.1: Synthesis of 11

About 1.3 g of lactonobionolactone was dissolved in 5 mL of methanol, then about 0.89 g (1.2 equivalents) of N-Boc-1,3-propanediamine was added to the reaction mixture. Now the reaction mixture was refluxed at 70° C. for 24 h. Then methanol was removed by rotavapour, the residue was washed with ethyl acetate and finally with chloroform. Then it was kept in high vacuum oven for overnight to get the pure and dry 11 with 72% yield. FT-IR (NaCl): 3341 cm⁻¹ (—OH str.), 2929 cm⁻¹ (—CH₂— asym. str.), 2888 cm⁻¹ (—CH₂— sym. str.), 1685 cm⁻¹ (Amide-I C═O str.), 1660 cm⁻¹ (Amide-II —NH— ben.). ¹H-NMR (400 MHz, DMSO-d₆) δ/ppm: 4.576 (d, 1H), 4.200-3.579 (m, 12H), 3.300 (t, 2H), 3.118 (t, 2H), 1.719 (Q, 2H), 1.446 (s, 9H). ¹³C-NMR (100 MHz, DMSO-d₆) δ/ppm: 171.96, 170.34, 103.15, 81.23, 73.23, 71.44, 69.13, 68.56, 62.27, 49.76, 36.21, 25.98. 21.02. HRMS: m/z 515.2489 (observed); 515.2452 (calculated for M+H⁺).

### Example 1.3.2: Synthesis of 12

About 1.35 g of 11 was dissolved in 5 mL of methanol and 5 mL of 4N HCl was added to it. Then it was kept for 5 h at room temperature. Then solvent was evaporated to get pure and dry compound 12 with 89% yield. FT-IR (NaCl): 3297 cm¹ (—OH and —NH₂ sym., asym. str.), 2932 cm⁻¹ (—CH₂— asym. str.), 2888 cm⁻¹ (—CH₂— sym. str.), 1685 cm⁻¹ (Amide-I C═O str.), 1648 cm⁻¹ (Amide-II —NH— ben.). ¹H-NMR (400 MHz, DMSO-d₆) δ/ppm: 4.572 (d, 1H), 4.411-3.576 (m, 12H), 3.352 (t, 2H), 3.303 (t, 2H), 1.721 (Q, 2H). ¹³C-NMR (100 MHz, DMSO-d₆) δ/ppm: 172.74, 103.12, 81.35, 73.30, 71.58, 69.10, 68.01, 62.84, 49.60, 36.05, 25.05. HRMS: m/z 415.1901 (observed); 415.1928 (calculated for M+H+).

### Example 1.4

### Synthesis of cationic-lipophilic-vancomycin-sugar conjugates (13-17)

About 1.0 equivalent of cationic-lipophilic-vancomycin analogue (6-10) was dissolved in 1:1 mixture of dry dimethyl formamide (1 mL) dry dimethyl sulfoxide (1 mL). To this two equivalents of compound 12 in 1 mL of dry dimethylformamide was added. The reaction mixture was cooled to about 0° C., and about 1.5 equivalents of 0.45 M HBTU solution in DMF was added followed by about 5.0 equivalents of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of cationic-lipophilic-vancomycin-sugar conjugates (13-17) with 60-70% yield. These conjugates were purified and characterized by ¹H-NMR and HR-MS (Table 1). The purification was done by preparative reverse phase HPLC using 0.1% Trifluoro acetic acid (TFA) in water/acetonitrile (0-100%) as mobile phase. C₁₈ column (10 mm diameter, 250 mm length) and UV detector (at 270 nm wave length) were used. The collected fractions, from HPLC were frozen by liquid N₂ and lyophilized in freeze dryer.

Compound 13: Yield; 70%. HR-MS: m/z 1031.1815 (observed), 1031.0054 (calculated for M+K)²⁺.

Compound 14: Yield; 63%. HR-MS: m/z 1045.8314 (observed), 1045.0317 (calculated for M+K)²⁺.

Compound 15: Yield; 67%. HR-MS: m/z 1058.3491 (observed), 1058.5545 (calculated for M+K)²⁺.

Compound 16: Yield; 65%. HR-MS: m/z 1073.8726 (observed), 1073.0843 (calculated for M+K)²⁺

Compound 17: Yield; 60%. HR-MS: m/z 1086.9958 (observed), 1087.1106 (calculated for M+K)²⁺

TABLE 1
HPLC retention times of vancomycin derivatives

| | Retention Time |
|---|---|
| Compound | (HPLC) [minutes] |
| Vancomycin | 7.934 |
| 6 | 10.347 |
| 7 | 11.430 |
| 8 | 12.170 |
| 9 | 13.473 |
| 10 | 14.232 |
| 13 | 9.865 |
| 14 | 10.192 |
| 15 | 11.317 |
| 16 | 12.325 |
| 17 | 13.879 |

### Example: 2

### In-Vitro Antibacterial Activity:

### Minimum Inhibitory Concentration (MIC):

All test compounds were assayed in a micro-dilution broth format. Stock solutions were made by serially diluting the compounds using autoclaved millipore water or broth media. The antibacterial activity of the compounds was determined againstmethicillin-resistant S. aureus (MRSA), vancomycin-intermediate-resistant S. aureus (VISA), vancomycin-sensitive E. faecium (VSE), vancomycin-resistant E. faecalis and vancomycin-resistant E. faecium (VRE). Bacteria, to be tested, were grown for about 10 h in the suitable media, MSSA, MRSA and VISA were grown in Yeast-dextrose broth (about 1 g of beef extract, about 2 g of yeast extract, about 5 g of peptone and about 5 g of NaCl in about 1000 mL of sterile distilled water (pH-7)). For solid media, about 5% agar was used along with above mentioned composition. VSE and VRE were cultured in Brain Heart Infusion broth (Himedia). The bacterial samples were freeze dried and stored at −80° C. About 5 μL of these stocks were added to about 3 mL of the nutrient broth or Brain Heart Infusion broth and the culture was grown for about 6 h at about 37° C. prior to the experiments. This 6 h grown culture gives about 10⁹ cfu/mL and this was determined by spread plating method. The 6 h grown culture was diluted to give effective cell concentration of about 10⁵ cfu/mL, which was then used for determining MIC. Compounds were serially diluted, in sterile water (2-fold dilution is employed) in a way that the working concentration was about 10 μM for MRSA, and VSE while for VRE and VISA it was about 100 μM. About 50 μL of these serial dilutions were added to the wells of 96 well plate followed by the addition of about 150 μL of bacterial solution. The plates were then incubated at about 37° C., 150 rpm in the incubator and O.D at 620 nm was recorded at an interval of about 24 h using TECAN (Infinite series, M200 pro) Plate Reader. Each concentration had triplicate values and the whole experiment was done at least twice and the MIC value was determined by taking the average of triplicate O. D. values for each concentration and plotting it against concentration. The data was then subjected to sigmoidal fitting. From the curve the MIC value was determined, as the point in the curve where the O.D. was similar to that of control having no bacteria.

The antibacterial activities of vancomycin derivatives and vancomycin against Staphylococci (MRSA and VISA) and Enterococci (VREm and VREs) were summarized in Table 2. The antibacterial activities of these derivatives were seen to be dependent on the length of alkyl chain appended to vancomycin. All the derivatives showed excellent antibacterial activity against both Staphylococci and Enterococci. Amongst these, the derivative 14 bearing 1-octyl-pyridinium and cyclic and acyclic form of sugar moiety showed the best activity against MRSA and VISA. Further, most exciting results were obtained in case of vancomycin-resistant Enterococci. When tested against highly pathogenic VREm, these derivatives exhibited minimum inhibitory concentration (MIC) in the range 0.09 to 0.37 μM. Again the derivative 14 showed MIC of 0.09 μM against VREm implying about >7500-fold more active than vancomycin.

In the present invention disclosure, we developed novel cationic-lipophilic-vancomycin-sugar conjugates using facile synthetic methodology. These derivatives showed strong, broad-spectrum antibacterial activity and about >7500-fold more active than parent drug, vancomycin against VRE. Thus, this strategy can be a promising approach to develop new generation of antibiotics to tackle multidrug-resistant bacterial infections.

TABLE 2
Antibacterial activities of vancomycin derivatives.

Minimum Inhibitory Concentration (μM)

| Compound | MRSA^{a} | VISA^{b} | VREm^{c} | VREs^{d} |
|---|---|---|---|---|
| Vancomycin | 0.78 | 8-12 | 750 | 250 |
| 7 | 0.1 | 0.11 | 2.9 | 6.2 |
| 8 | 0.12 | 0.13 | 1.83 | 3.3 |
| 9 | 0.25 | 0.25 | 0.98 | 1.5 |
| 14 | 0.09 | 0.1 | 0.09 | 1.36 |
| 15 | 0.2 | 0.09 | 0.3 | 1.3 |
| 16 | 0.28 | 0.31 | 0.37 | 1.11 |

^{a}Methicillin-resistant S. aureus (ATCC 33591).
^{b}Vancomycin intermediate resistant S. aureus.
^{c}Vancomycin-resistant E. faecium (ATCC 51559).
^{d}Vancomycin-resistant E. faecalis (VanA, ATCC 51575).

### Example: 3

In-vivo activity against methicillin-resistant vancomycin intermediate S. aureus (MR-VISA):

Mouse Neutropenic Thigh Infection Model:

About six-week-old, female CD-1 mice (weight, ˜19-24 g) were used for the experiments. The mice were rendered neutropenic (˜100 neutrophils/ml) by injecting two doses of cyclophosphamide intraperitoneally 4 days (150 mg/kg) and 1 day (100 mg/kg) before the infection experiment. 50 μL of ˜10 CFU/mouse concentration of the bacterial inoculum (MR-VISA) was injected into the thigh. One hour after inoculation, animals were treated intravenously with saline, vancomycin, linezolid and compound 14 at 12 mg/kg body weight. At 24 h post first treatment, cohorts of animals were euthanized (using ether) and the thighs were collected aseptically. The thigh was weighed (0.7 g-0.9 g) and placed into 10 ml of sterile saline and homogenized. The dilutions of the homogenate were plated onto agar plates, which were incubated overnight at 37° C. The bacterial titer was expressed as log₁₀ CFU/g of thigh weight.

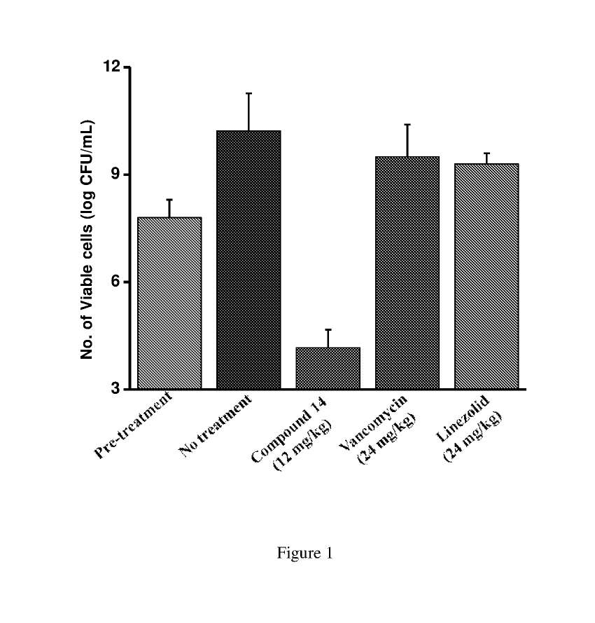

The in-vivo efficacy of compound 14 in comparison with linezolid and vancomycin against MR-VISA was shown in (FIG. 1). The bacterial density taken from animals prior to initiation of dosing was determined to be 7.8 log₁₀ CFU/g. After 24 h of the initial treatment, antibacterial activity was determined by finding the bacterial titer in the infected thighs. Vancomycin and linezolid resulted in minimal or no activity compared to vehicle treated mice (saline). In contrast, compound 14 showed excellent efficacy, where it produced ˜6.0 log₁₀ CFU/g reduction in bacterial count from the vehicle treated mice (ED_{3-log kill})

### Advantage

The above mentioned implementation examples as described on this subject matter and its equivalent thereof have many advantages, including those which are described.

The disclosed compounds and/or derivatives in the present invention can provide better interaction with the cell wall of the bacteria through improved hydrogen bonding interactions. This increased association with bacterial cell wall precursors can serve as to inhibit the cell wall biosynthesis in both sensitive and resistant bacteria.

Although the subject matter has been described in considerable details with reference to certain preferred embodiments thereof, other embodiment are possible. As such, the spirit and scope of the appended claims should not be limited to the description of the preferred embodiments contained therein.

### REFERENCES

1) N. Woodford, D. M. Livermore, Infections caused by Gram-positive bacteria: A review of the global challenge. J. Infect. 59, S4-S16 (2009).
2) R. Nagarajan, Glycopeptide Antibiotics Ed. Marcel Dekker, New York, 1994.
3) D. Kahne, C. Leimkuhler, W. Lu, C. T. Walsh, Glycopeptide and lipopeptide antibiotics. Chem. Rev. 105, 425-448 (2005).
4) V. Yarlagadda, P. Akkapeddi, G. B. Manjunath, J. Haldar, Membrane active vancomycin analogues: A novel strategy to combat bacterial resistance, J. Med. Chem. 57, 4558-4568 (2014).
5) J. Haldar, V. Yarlagadda, P. Akkapeddi, Cationic antibacterial composition. WO 2013072838 A1
6) J. Haldar, V. Yarlagadda, G. B. Manjunath, M. M. Konai, Vancomycin-sugar conjugates and uses thereof. Patent application no. 4314/CHENP/2013.
7) D. Chu, M. N. Preobrazhenskaya, S. S. Printsevskaya, E. N. Olsufyeva, Semi-synthetic glycopeptides with antibiotic activity. U.S. Pat. No. 7,632,918 B2, (2007).

1-20. (canceled)

21. A compound of Formula I 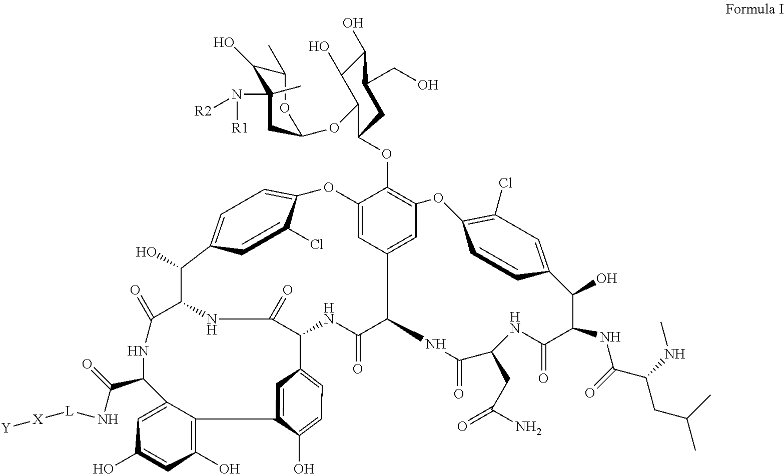
or its stereoisomers, prodrugs and pharmaceutically acceptable salts thereof,
wherein R¹ is selected from hydrogen, substituted or unsubstituted C₁-C₁₈ aliphatic radical or substituted or unsubstituted C₅-C₁₈ aromatic radical;
R² is C₁-C₁₈ aliphatic radical, substituted with trialkylamino, heteroaryl or heterocyclyl; wherein heteroaryl, and heterocyclyl are independently substituted with upto four substituents selected from hydrogen, substituted or unsubstituted C₁-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₁₈ aromatic radical;
X is NH and O; and
Y is selected from the group consisting of cyclic monosaccharide, cyclic disaccharide, acyclic monosaccharide, acyclic disaccharide, and combinations thereof; or
a compound of Formula Ia 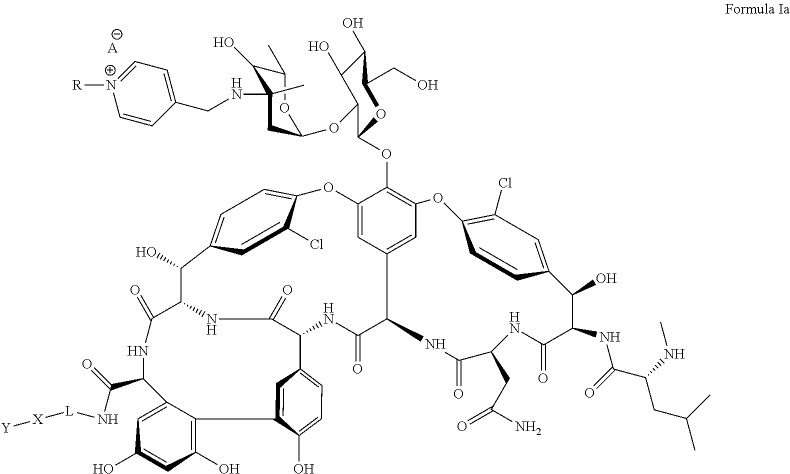
or its stereoisomers, prodrugs and pharmaceutically acceptable salts thereof:
R is selected from hydrogen, substituted or unsubstituted C₂-C₁₈ aliphatic radical or substituted or unsubstituted C₅-C₁₈ aromatic radical;
L is selected from substituted or unsubstituted C₂-C₁₈ aliphatic radical or substituted or unsubstituted C₅-C₈ aromatic radical;
X is NH and O; and
Y is selected from the group consisting of cyclic monosaccharide, cyclic disaccharide, acyclic monosaccharide, acyclic disaccharide, and combinations thereof;
A^{⊖} is negatively charged counter anion.

22. The compound of claim 21, wherein R is selected from the group consisting of hydrogen, a C₂-C₁₈ alkyl, a C₅-C₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl, 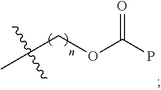
wherein alkyl, alkenyl, alkynyl, cycloalkyl, cycl 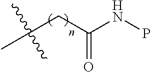
oalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;
P is a C₂-C₁₈ aliphatic radical and n is an integer ranging from 1 to 4,
L is a C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;
X is NH and O;
Y is selected from the group consisting of cyclic monosaccharide, cyclic disaccharide, acyclic monosaccharide, acyclic disaccharide, and combinations thereof; and
A^{⊖} is negatively charged counter anion.

23. The compound of claim 21, wherein Y is selected from the group consisting of

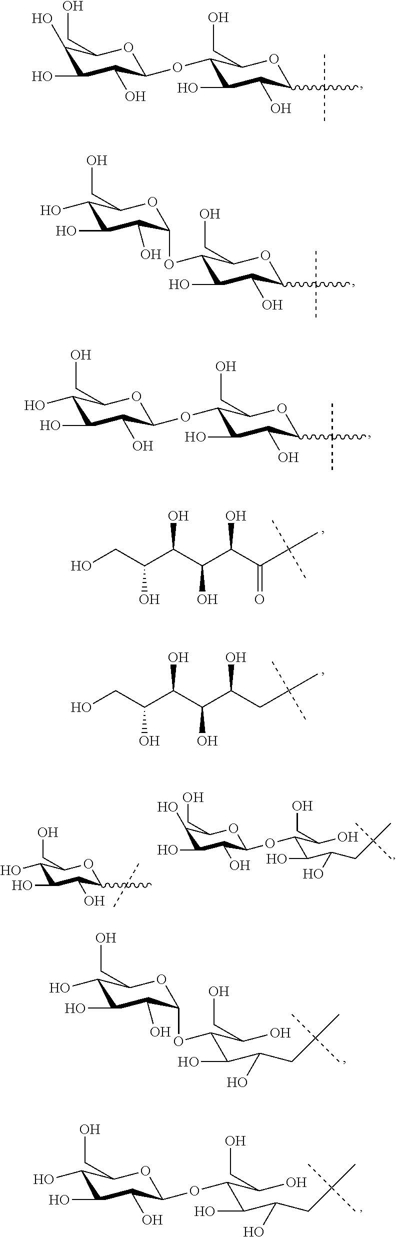

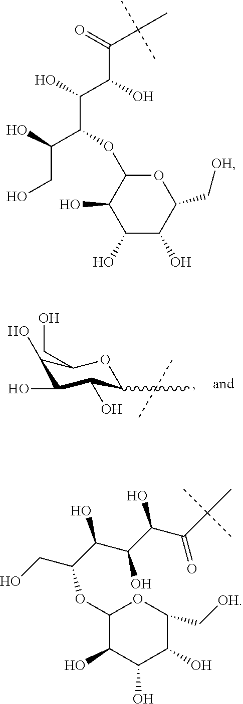

24. The compound of claim 21, wherein Y is selected from the group consisting of

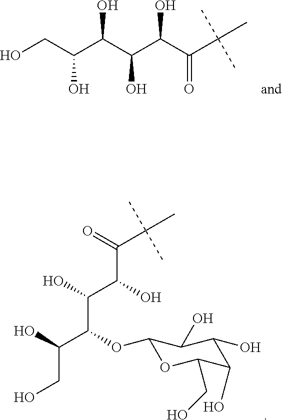

25. The compound of claim 21, wherein
R is selected from the group consisting of hydrogen, substituted or unsubstituted C₂-C₁₈ aliphatic radical, and substituted or unsubstituted C₅-C₁₈ aromatic radical;
L is C₂-C₁₈ aliphatic radical;
X is NH;
Y is selected from the group consisting of

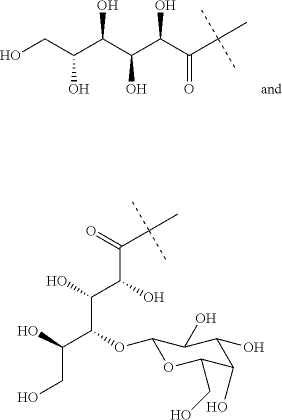

26. The compound of claim 21, wherein 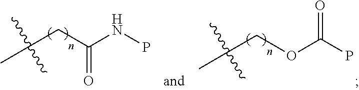
R is selected from and
L is C₂-C₁₈ aliphatic radical;
X is NH;
Y is selected from the group consisting of 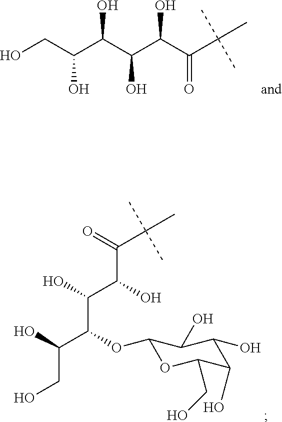
and
P is a C₂-C₁₈ aliphatic radical and n is an integer ranging from 1 to 4.

27. The compound of claim 21, wherein
R is C₆-C₁₈ aliphatic radical;
L is C₃ aliphatic radical;
X is NH; and
Y is selected from the group consisting of

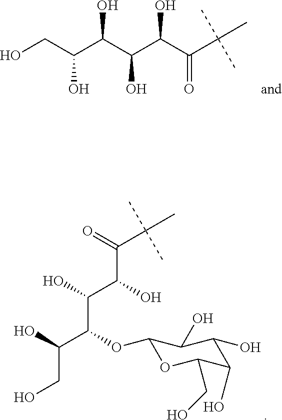

28. The compound of claim 21, wherein
R is selected from the group consisting of hydrogen, a C₂-C₁₈ alkyl, a C₅-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl, 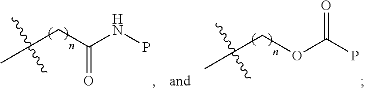
wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with up to four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;
L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;
X is NH; and
Y is selected from the group consisting of

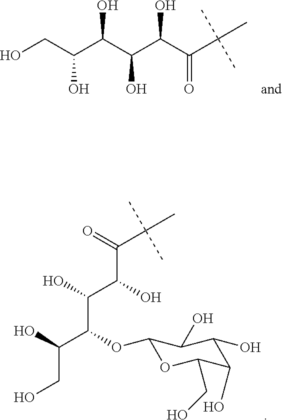

29. The compound of claim 21, wherein
R is selected from 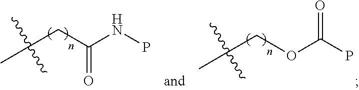
L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;
X is NH;
Y is selected from the group consisting of 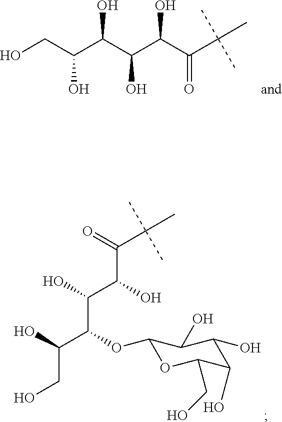
P is a C₂-C₁₈ aliphatic radical and n is an integer ranging from 1 to 4,

30. The compound of claim 21, wherein
R is C₆-C₁₈ alkyl;
L is C₂-C₆ alkyl, alkenyl, alkynyl, haloalkyl, hydroxyalkyl, carboxyalkyl, cycloalkyl, cycloalkylalkyl, heterocyclyl, heterocyclylalkyl; wherein alkyl, alkenyl, alkynyl, cycloalkyl, cycloalkylalkyl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;
X is NH; and
Y is selected from the group consisting of

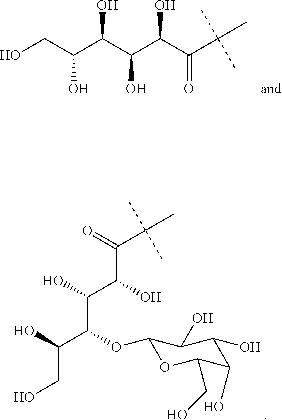

31. The compound of claim 21, wherein the compound is selected from the group consisting of:

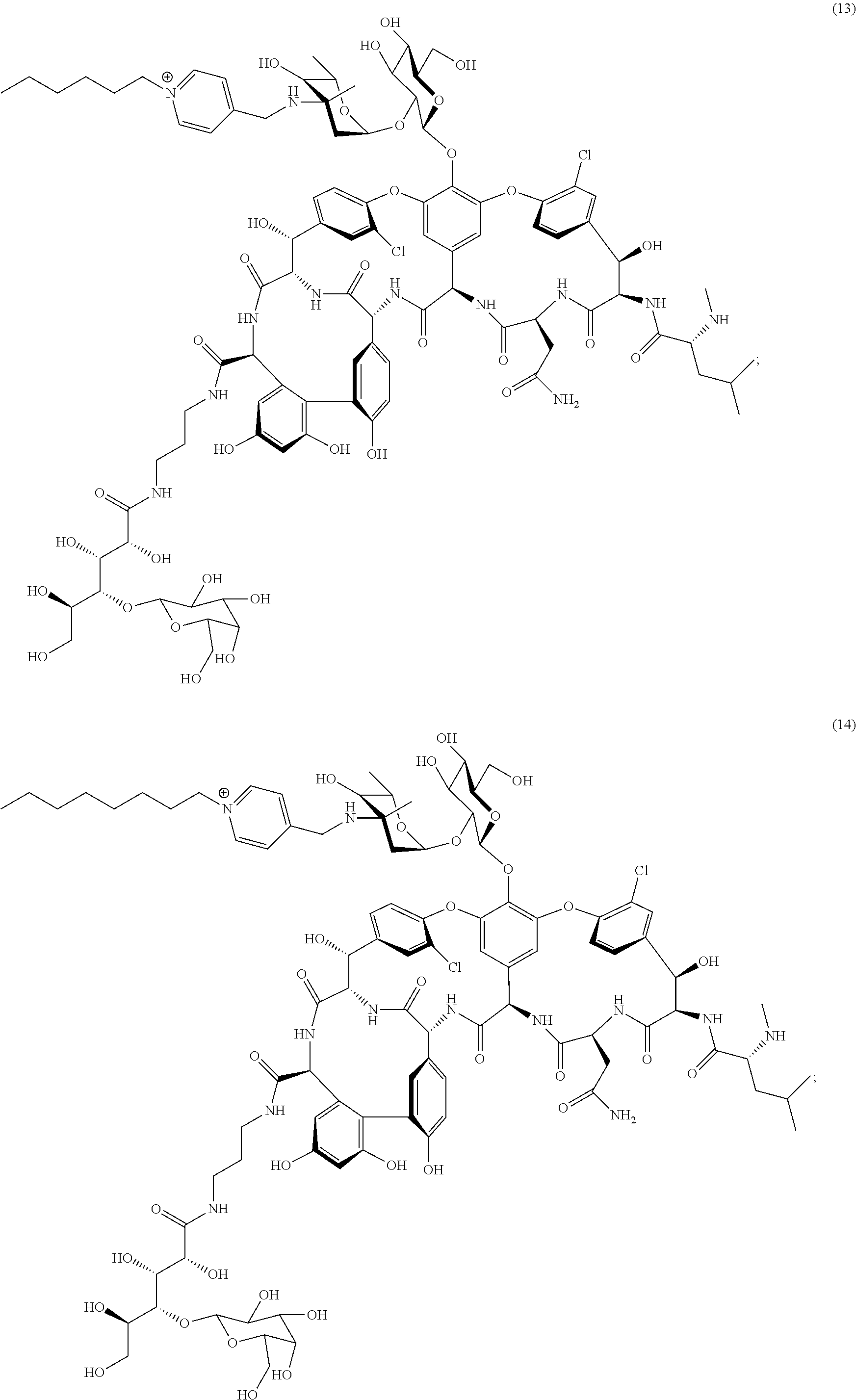

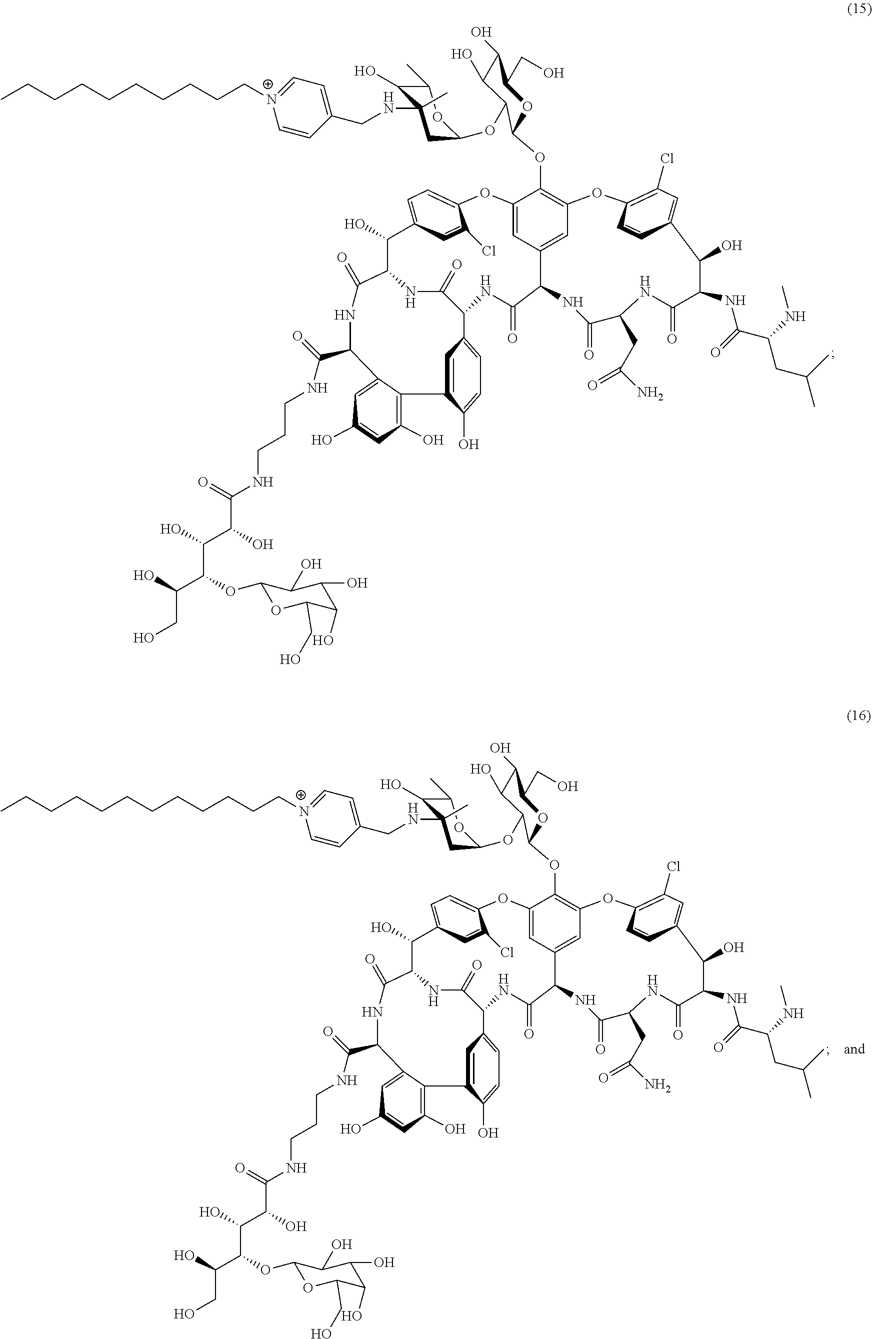

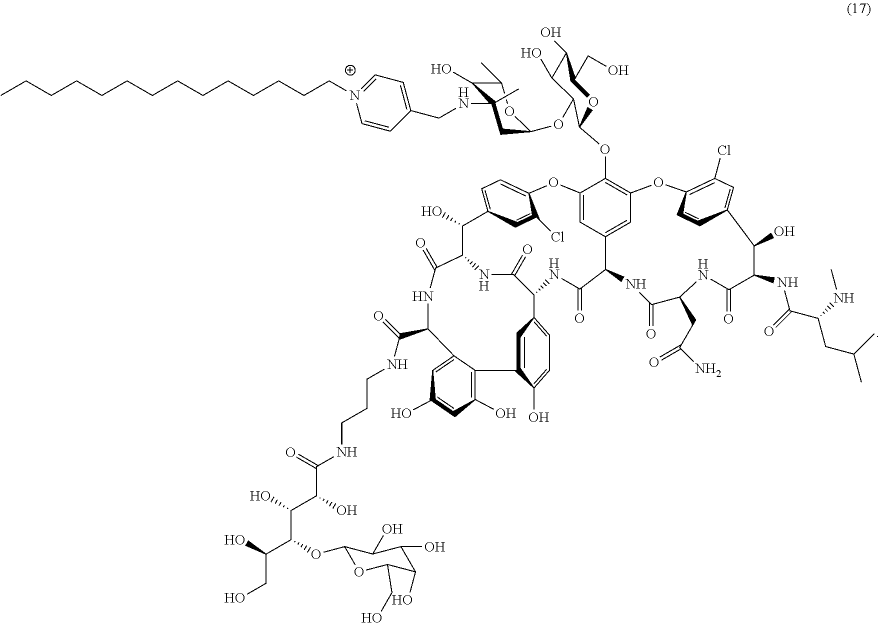

32. A method of treating a bacterial infection, comprising administering the compound of claim 21.

33. The method of claim 32, wherein the bacterium is a gram positive bacterium.

34. The method of claim 32, wherein the bacterium is a vancomycin-resistant bacterium or a methicillin-resistant bacterium.

35. The method of claim 34, wherein the bacterium is a vancomycin-resistant Staphylococcus aureus, a vancomycin-resistant Enterococcus faecium, or a methicillin-resistant Staphylococcus aureus.

36. A pharmaceutical composition comprising the compound of claim 21 and a pharmaceutically acceptable carrier.

37. The pharmaceutical composition of claim 36, and further comprising one or more other pharmaceutical compositions.
